(19) United States
(12) Patent Application Publication
(10) Pub. No.: US 2012/0197175 A1
(43) Pub. Date: Aug. 2, 2012

(54) METHODS, SYSTEMS AND APPARATUS FOR RELIEVING PRESSURE IN AN ORGAN

(75) Inventors: Christopher Horvath, Desert Hot Springs, CA (US); Ronald D. Bache, Mission Viejo, CA (US); Laszlo O. Romoda, San Clemente, CA (US); Dao-Yi Yu, City Beach (AU); Cory Anderson, Alpharetta, GA (US); Roelof Trip, Suwanee, GA (US); Ying Yang, Union City, CA (US); Hoang Van Nguyen, San Jose, CA (US); Surag Mantri, Sunnyvale, CA (US); Er-Ning Su, City Beach (AU); Stephen Cringle, Shenton Park (AU); James McCrea, San Carlos, CA (US); Daniel Mufson, Napa, CA (US); Colin Tan, Sunnyvale, CA (US); Richard L. Lindstrom, Bloomington, MN (US)

(73) Assignee: AQUESYS, INC., Irvine, CA (US)

(21) Appl. No.: 13/314,927

(22) Filed: Dec. 8, 2011

Related U.S. Application Data
(63) Continuation-in-part of application No. 11/771,805, filed on Jun. 29, 2007.
Application No. 11/771,805 is a continuation-in-part of application No. 12/946,351, filed on Nov. 15, 2010.
(60) Provisional application No. 60/806,402, filed on Jun. 30, 2006.

(51) Int. Cl. A61F 9/00

(52) U.S. Cl. 604/8

(57) ABSTRACT

The invention generally relates to shunts in which at least a portion of the body includes a drug.

### CROSS-REFERENCE TO RELATED APPLICATION

This application is a continuation-in-part of U.S. nonprovisional patent application Ser. No. 11/771,805, filed Jun. 29, 2007, which claims the benefit of and priority to U.S. provisional patent application Ser. No. 60/806,402, filed Jun. 30, 2006. This application is also a continuation-in-part of U.S. nonprovisional patent application Ser. No. 12/946,351, filed Nov. 15, 2010. The entire contents of each application is hereby incorporated by reference.

### FIELD OF THE INVENTION

The invention generally relates to shunts in which at least a portion of the body includes a drug.

### BACKGROUND

Glaucoma is a disease of the eye that affects millions of people. Glaucoma is associated with an increase in intraocular pressure resulting either from a failure of a drainage system of an eye to adequately remove aqueous humor from an anterior chamber of the eye or overproduction of aqueous humor by a ciliary body in the eye. Build-up of aqueous humor and resulting intraocular pressure may result in irreversible damage to the optic nerve and the retina, which may lead to irreversible retinal damage and blindness.

Glaucoma may be treated in a number of different ways. One manner of treatment involves delivery of drugs such as beta-blockers or prostaglandins to the eye to either reduce production of aqueous humor or increase flow of aqueous humor from an anterior chamber of the eye. Glaucoma may also be treated by surgical intervention that involves placing a shunt in the eye to result in production of fluid flow pathways between an anterior chamber of an eye and various structures of the eye involved in aqueous humor drainage (e.g., Schlemm's canal, the sclera, or the subconjunctival space). Such fluid flow pathways allow for aqueous humor to exit the anterior chamber.

One problem with implantable shunts is that they are composed of a rigid material, e.g., stainless steel, that does not allow the shunt to react to movement of tissue surrounding the eye. Consequently, existing shunts have a tendency to move after implantation, affecting ability of the shunt to conduct fluid away from the anterior chamber of the eye. To prevent movement of the shunt after implantation, certain shunts are held in place in the eye by an anchor that extends for a body of the shunt and interacts with the surrounding tissue. Such anchors result in irritation and inflammation of the surrounding tissue.

Another problem with implantable shunts is that they may become clogged, preventing aqueous humor from exiting the anterior chamber, and resulting in re-occurrence of fluid build-up in the eye. Such a problem may only be fixed by surgical intervention.

Additionally, existing implantable shunts do not effectively regulate fluid flow from the anterior chamber, i.e., fluid flow is passive from the anterior chamber to a drainage structure of the eye and is not regulated by the shunt. If fluid flows from the anterior chamber at a rate greater than it can be produced in the anterior chamber, the chamber will collapse, resulting in significant damage to the eye and requiring surgical intervention to repair. If fluid flow from the eye is not great enough, pressure in the anterior chamber will not be relieved, and damage to the optic nerve and the retina may still occur.

### SUMMARY

The invention generally provides improved shunts that facilitate drainage of fluid from an organ. Particularly, shunts of the invention address and solve the above described problems by providing shunts that are impregnated or coated with a drug or combination of drugs that regulate the body's response to the implantation of the shunt and the subsequent healing process.

In certain aspects, the invention generally provides drug impregnated or coated shunts composed of a material that has an elasticity modulus that is compatible with an elasticity modulus of tissue surrounding the shunt. In this manner, shunts of the invention are flexibility matched with the surrounding tissue, and thus will remain in place after implantation without the need for any type of anchor that interacts with the surrounding tissue. Consequently, shunts of the invention will maintain fluid flow away for an anterior chamber of the eye after implantation without causing irritation or inflammation to the tissue surrounding the eye.

Although discussed in the context of the eye, the elasticity modulus of the shunt may be matched to the elasticity modulus of any tissue. Thus, shunts of the invention may be used to drain fluid from any organ. In particular embodiments, the organ is an eye. Shunts of the invention may define a flow path from an area of high pressure in the eye (e.g., an anterior chamber) to an area of lower pressure in the eye (e.g., intra-Tenon's space, the subconjunctival space, the episcleral vein, the suprachoroidal space, and Schlemm's canal).

In other aspects, the invention generally provides drug impregnated or coated shunts in which a portion of the shunt is composed of a flexible material that is reactive to pressure, i.e., an inner diameter of the shunt fluctuates depending upon the pressures exerted on that portion of the shunt. Thus, the flexible portion of the shunt acts as a valve that regulates fluid flow through the shunt. After implantation, intraocular shunts have pressure exerted upon them by tissues surrounding the shunt (e.g., scleral tissue) and pressure exerted upon them by aqueous humor flowing through the shunt. When the pressure exerted on the flexible portion of the shunt by the surrounding tissue is greater than the pressure exerted on the flexible portion of the shunt by the fluid flowing through the shunt, the flexible portion decreases in diameter, restricting flow through the shunt. The restricted flow results in aqueous humor leaving the anterior chamber at a reduced rate.

When the pressure exerted on the flexible portion of the shunt by the fluid flowing through the shunt is greater than the pressure exerted on the flexible portion of the shunt by the surrounding tissue, the flexible portion increases in diameter, increasing flow through the shunt. The increased flow results in aqueous humor leaving the anterior chamber at an increased rate.

The flexible portion of the shunt may be any portion of the shunt. In certain embodiments, the flexible portion is a distal portion of the shunt. In certain embodiments, the entire shunt is composed of the flexible material.

Other aspects of the invention generally provide drug impregnated or coated multi-port shunts. Such shunts reduce probability of the shunt clogging after implantation because fluid can enter or exit the shunt even if one or more ports of the shunt become clogged with particulate. In certain embodiments, the shunt includes a hollow body defining a flow path and more than two ports, in which the body is configured such that a proximal portion receives fluid from the anterior chamber of an eye and a distal portion directs the fluid to a location of lower pressure with respect to the anterior chamber.

The shunt may have many different configurations. In certain embodiments, the proximal portion of the shunt (i.e., the portion disposed within the anterior chamber of the eye) includes more than one port and the distal portion of the shunt (i.e., the portion that is located in an area of lower pressure with respect to the anterior chamber such as intra-Tenon's space, the subconjunctival space, the episcleral vein, the suprachoroidal space, or Schlemm's canal) includes a single port. In other embodiments, the proximal portion includes a single port and the distal portion includes more than one port. In still other embodiments, the proximal and the distal portions include more than one port.

The ports may be positioned in various different orientations and along various different portions of the shunt. In certain embodiments, at least one of the ports is oriented at an angle to the length of the body. In certain embodiments, at least one of the ports is oriented 90° to the length of the body.

The ports may have the same or different inner diameters. In certain embodiments, at least one of the ports has an inner diameter that is different from the inner diameters of the other ports.

Other aspects of the invention generally provide drug impregnated or coated shunts with overflow ports. Those shunts are configured such that the overflow port remains closed until there is a pressure build-up within the shunt sufficient to force open the overflow port. Such pressure build-up typically results from particulate partially or fully clogging an entry or an exit port of the shunt. Such shunts reduce probability of the shunt clogging after implantation because fluid can enter or exit the shunt by the overflow port even in one port of the shunt becomes clogged with particulate.

In certain embodiments, the shunt includes a hollow body defining an inlet configured to receive fluid from an anterior chamber of the eye and an outlet configured to direct the fluid to a location of lower pressure with respect to the anterior chamber, the body further including at least one slit. The slit may be located at any place along the body of the shunt. In certain embodiments, the slit is located in proximity to the inlet. In other embodiments, the slit is located in proximity to the outlet. In certain embodiments, there is a slit in proximity to both the inlet and the outlet of the shunt.

In certain embodiments, the slit has a width that is substantially the same or less than an inner diameter of the inlet. In other embodiments, the slit has a width that is substantially the same or less than an inner diameter of the outlet. Generally, the slit does not direct the fluid unless the outlet is obstructed. However, the shunt may be configured such that the slit does direct at least some of the fluid even if the inlet or outlet is not obstructed.

In other aspects, the invention generally provides drug impregnated or coated shunts having a variable inner diameter. In particular embodiments, the diameter increases from inlet to outlet of the shunt. By having a variable inner diameter that increases from inlet to outlet, a pressure gradient is produced and particulate that may otherwise clog the inlet of the shunt is forced through the inlet due to the pressure gradient. Further, the particulate will flow out of the shunt because the diameter only increases after the inlet.

In certain embodiments, the shunt includes a hollow body defining a flow path and having an inlet configured to receive fluid from an anterior chamber of an eye and an outlet configured to direct the fluid to a location of lower pressure with respect to the anterior chamber, in which the body further includes a variable inner diameter that increases along the length of the body from the inlet to the outlet. In certain embodiments, the inner diameter continuously increases along the length of the body. In other embodiments, the inner diameter remains constant along portions of the length of the body. Exemplary locations of lower pressure include the intra-Tenon's space, the subconjunctival space, the episcleral vein, the subarachnoid space, and Schlemm's canal.

Shunts of the invention may be coated or impregnated with at least one drug, e.g., pharmaceutical and/or biological agent or a combination thereof. The pharmaceutical and/or biological agent may coat or impregnate an entire exterior of the shunt, an entire interior of the shunt, or both. Alternatively, the pharmaceutical and/or biological agent may coat and/or impregnate a portion of an exterior of the shunt, a portion of an interior of the shunt, or both. Methods of coating and/or impregnating a medical device with a pharmaceutical and/or biological agent are shown, for example, in Darouiche (U.S. Pat. Nos. 7,790,183; 6,719,991; 6,558,686; 6,162,487; 5,902,283; 5,853,745; and 5,624,704). The content of each of these references is incorporated by reference herein its entirety.

In certain embodiments, the exterior portion of the shunt that resides in the anterior chamber after implantation (e.g., about 1 mm of the proximal end of the shunt) is coated and/or impregnated with the pharmaceutical or biological agent. In other embodiments, the exterior of the shunt that resides in the scleral tissue after implantation of the shunt is coated and/or impregnated with the pharmaceutical or biological agent. In other embodiments, the exterior portion of the shunt that resides in the area of lower pressure (e.g., the intra-Tenon's space or the subconjunctival space) after implantation is coated and/or impregnated with the pharmaceutical or biological agent. In embodiments in which the pharmaceutical or biological agent coats and/or impregnates the interior of the shunt, the agent may be flushed through the shunt and into the area of lower pressure (e.g., the intra-Tenon's space or the subconjunctival space).

Any pharmaceutical and/or biological agent or combination thereof may be used with shunts of the invention. The pharmaceutical and/or biological agent may be released over a short period of time (e.g., seconds) or may be released over longer periods of time (e.g., days, weeks, months, or even years). Exemplary agents include anti-mitotic pharmaceuticals such as Mitomycin-C or 5-Fluorouracil, anti-VEGF (such as Lucintes, Macugen, Avastin, VEGF or steroids).

The shunts discussed above and herein are described relative to the eye and, more particularly, in the context of treating glaucoma and solving the above identified problems relating to intraocular shunts. Nonetheless, it will be appreciated that shunts described herein may find application in any treatment of a body organ requiring drainage of a fluid from the organ and are not limited to the eye.

### BRIEF DESCRIPTION OF THE DRAWINGS

FIG. 1, depicts, in general, a method for implanting a shunt, showing in cross section, the distal end of an implantation apparatus;

FIG. 2 is an enlarged, schematic view of the distal end of one embodiment of an implantation apparatus described herein;

FIG. 3 is a perspective view of one embodiment of a handheld implantation apparatus with the door opened and a needle assembly installed therein;

FIG. 4 is a top view of the apparatus of FIG. 3 with front door removed;

FIG. 5 is an exploded view of the system for implanting a shunt including the apparatus FIG. 3;

FIG. 6 is a perspective view of the distal end of the apparatus of FIG. 3 with the needle assembly separated therefrom;

FIG. 7 is an enlarged perspective view of the needle assembly of FIG. 6;

FIG. 8 is an exploded view of the needle assembly of FIG. 7;

FIG. 9(a)-(f) are schematic views of the implantation apparatus of FIG. 3 showing the plunger, guidewire and needle arms in different positions during the positioning and/or implantation steps as they correspond to the positions of the plunger, guidewire, needle and shunt within the eye;

FIG. 10 is a perspective view of another embodiment of an implantation apparatus with the needle assembly installed therein;

FIG. 11 is a perspective view of the implantation apparatus of FIG. 10 and the disposable needle assembly in its extended state and separated therefrom;

FIG. 12 is a side view of another embodiment of a handheld and manually operated implantation apparatus;

FIG. 13 is a side view of still another embodiment of a handheld and manually operated implantation apparatus;

FIG. 14 is an enlarged side view of the needle assembly of the apparatus of FIG. 13;

FIG. 15 is a perspective view of a syringe type, manually operated, handheld implantation apparatus;

FIG. 16 is a schematic illustration of a method and apparatus for making a gelatin microfistula shunt in the form of a tube;

FIG. 17 is a schematic illustration an alternative embodiment of an apparatus for making a gelatin microfistula tube;

FIG. 18 is a perspective view of an apparatus for making a plurality of microfistula gelatin tubes.

FIG. 19 is a front view of a graduated needle inserted into the eye of a patient;

FIG. 20 is a front view showing a transpupil shunt insertion and placement;

FIG. 21 is a schematic view showing an ipsilateral tangential shunt insertion and placement;

FIG. 22(a)-(d) depicts a series of steps showing an ipsilateral normal shunt insertion and placement using a U-shaped or otherwise arcuate needle;

FIG. 23 is a perspective view of a U-shaped needle of the type shown in the method of insertion and placement shown in FIGS. 22(a)-(d);

FIG. 24 is a front view of the U-shaped needle of FIG. 23;

FIG. 25 is a side view of a needle having a bend at its distal end portion including the guidewire and shunt inserted therein;

FIG. 26 is a cross-sectional view of the needle, guidewire, plunger and shunt of the needle distal end portion of FIG. 25;

FIG. 27 is a side view of the needle, the plunger or guidewire of FIG. 25, wherein a portion of the plunger or guidewire facilitates bending of the same;

FIG. 28 is a perspective view of a cylindrical shunt including a tapered end;

FIG. 29 is a perspective view of a cylindrical shunt including retaining tabs for limiting migration of the shunt;

FIG. 30 is an end view of the tabbed shunt of FIG. 29;

FIG. 31 is a perspective view of a cylindrical shunt including centrally located barbs to limit migration of the implanted shunt;

FIG. 32 is a perspective view of a cylindrical shunt including barbs located at one of the implanted shunt to limit migration thereof;

FIG. 33 shows the tabbed shunt of FIGS. 29-30 inserted within the eye of the patient.

### DESCRIPTION OF THE EMBODIMENTS

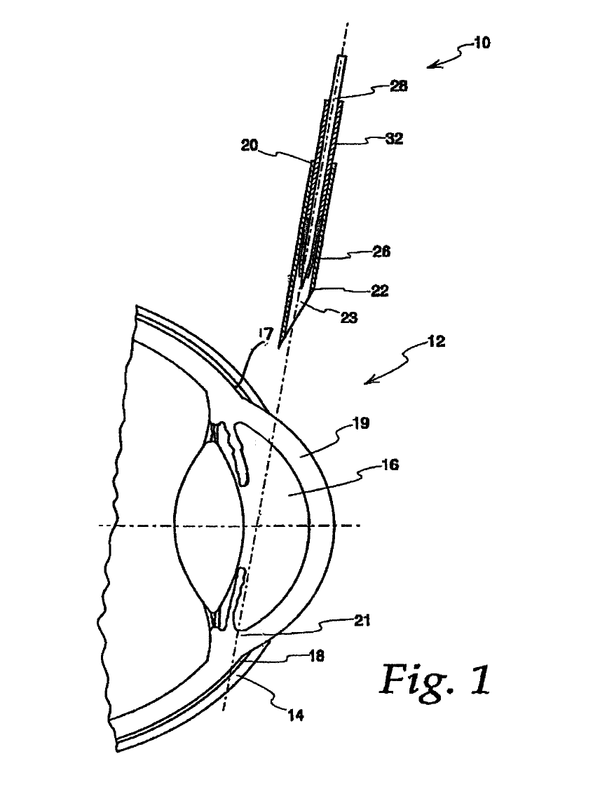

Methods and apparatus for delivering and implanting bioabsorbable tubes or shunts are generally disclosed in U.S. Pat. Nos. 6,544,249 and 6,007,511, both of which have been previously incorporated by reference in their entireties. As set forth therein, and also with reference to FIG. 1, an implantation apparatus 10 is used to deliver and implant a small micro-sized bioabsorbable tube i.e., the microfistula tube 26, to an area between the anterior chamber 16 and the sub-conjunctival space 18 of the eye 12. The implanted microfistula tube 26 provides a shunt that continuously drains aqueous humor from anterior chamber 16 at a desired rate. Microfistula tube 26 remains implanted in the eye, and eventually dissolves.

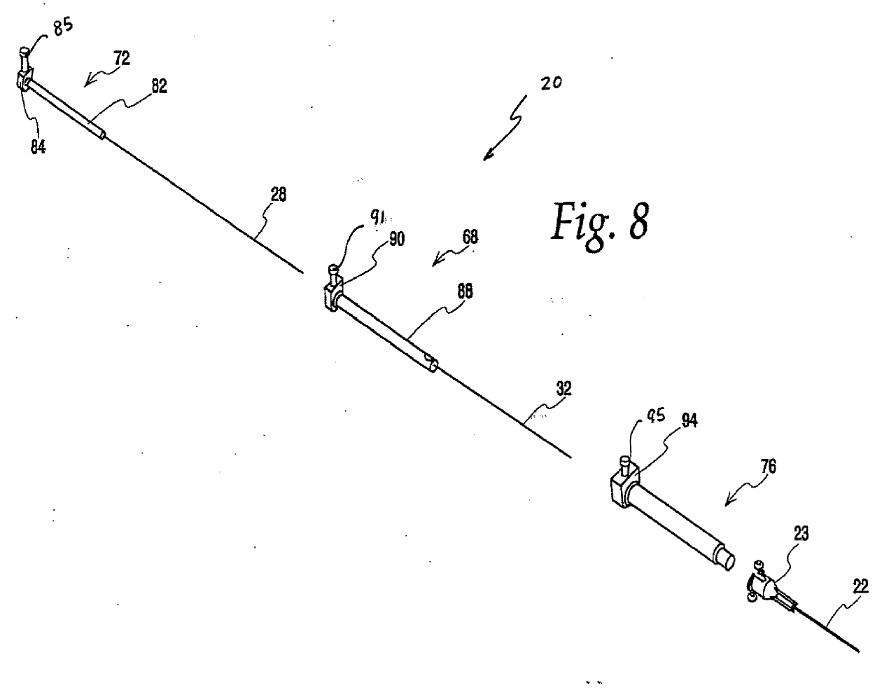

FIG. 1 illustrates the distal (i.e., “working end”) end of the apparatus 10 (including the microfistula tube 26) as it approaches the eye 12 as described in U.S. Pat. No. 6,544,249. Unlike current trabeculectomy procedures, in accordance with the method shown in FIG. 1, needle 22 housing microfistula tube 26 approaches and enters the eye through cornea 19 (ab interno) and not through the conjunctiva 14 (ab externo). This prevents damage to the conjunctiva, improves healing time and reduces the risk of complications that may result from other surgical techniques of the prior art (e.g., trabeculectomy). As further shown and described in U.S. Pat. No. 6,544,249 and in FIG. 1, hollow needle 20 is introduced through the cornea 19 and is advanced across the anterior chamber 16 (as depicted by the broken line) in what is sometimes referred to as a transpupil implant insertion. Shunt 26 is eventually implanted in the area spanning the sclera 21, anterior chamber 16 and the sub-conjunctival space 18 (see also FIG. 8 of U.S. Pat. No. 6,544,249).

The methods, systems, apparatus and shunts described herein likewise utilize a hollow needle and a bioabsorbable shunt delivered by the needle ab interno through the cornea 19 or the surgical limbus 17. As used herein, the term “shunt” includes hollow microfistula tubes similar to the type generally described in U.S. Pat. No. 6,544,249 as well as other structures that include one or more flow paths therethrough.

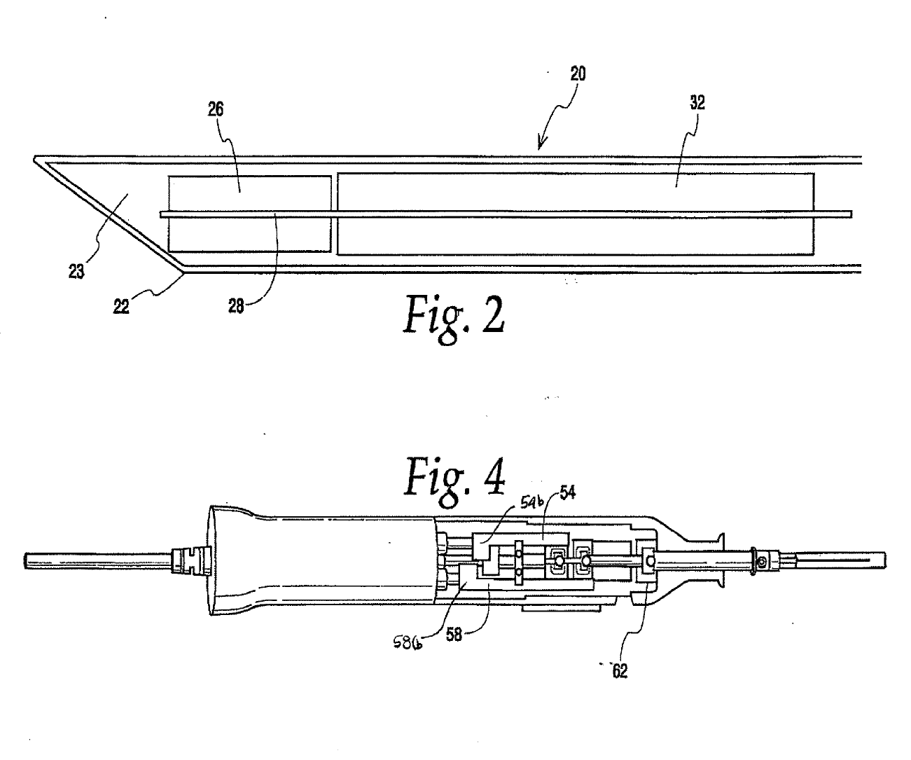

Turning now to a discussion of the methods, systems, apparatus and shunts that embody the present invention, as generally shown in FIG. 2, the working end of implantation apparatus is provided as a needle assembly 20 that includes a hollow needle 22 defining an inner chamber 23 and terminating in a sharpened tip. Placed within inner chamber 23 of the hollow needle 22 is a cylindrical inner tube or plunger 32 that is coaxial with needle 22. In the loaded and ready to use condition, shunt 26 is also placed or otherwise disposed within the hollow chamber 23 of needle 22 and is distally located relative to plunger 32. Both shunt 26 and plunger 32 may be placed over and supported by optional guidewire 28. As described in U.S. Pat. No. 6,544,249 and in this disclosure, through relative movement of needle 22, plunger 32, guidewire 28, and shunt 26 can be implanted into eye 12. As noted above, guidewire 28 is optional and may be omitted where placement and advancement of shunt 26 does not require one.

As will be described in greater detail below, shunt 26 may be delivered to and implanted within the desired location of the eye in any one of several different ways. The method of implantation (and system) may be fully automated, partially automated (and, thus, partially manual) or completely manual. For example, in a fully automated procedure, shunt 26 may be delivered by robotic implantation whereby a surgeon controls the advancement of needle 22, plunger 32, optional guidewire 28 and, as a result, shunt 26 by remotely controlling a robot. In such fully automated, remotely controlled procedures, the surgeon's hands typically do not contact implantation apparatus 10 during the surgical procedure.

Alternatively, shunt 26 may be delivered to the desired area of the eye with a “handheld” implantation apparatus, embodiments of which are shown in FIGS. 2-15 and described below. In one example of a handheld implantation apparatus, discussed in more detail below, movement of the shunt 26, needle 22, and plunger 32 and optional guidewire 28 may be controlled remotely by an operator using a microprocessor-based device i.e., “controller,” while implantation apparatus 10 is physically held by the surgeon. Insertion of the needle into the eye as well as certain repositioning or adjusting steps may be performed manually by the surgeon.

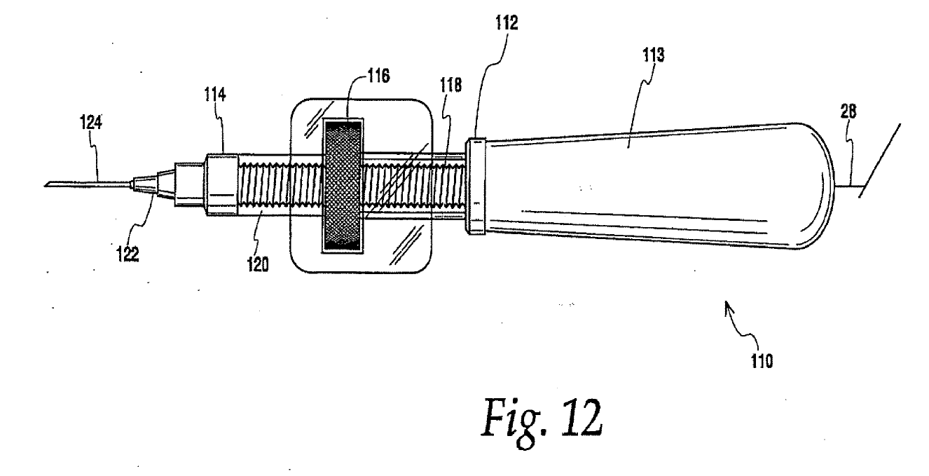

In the case of fully manual apparatus and methods, which are also discussed below and shown in FIGS. 12-15, all of the positioning, repositioning, adjusting and implantation steps are performed manually by the surgeon.

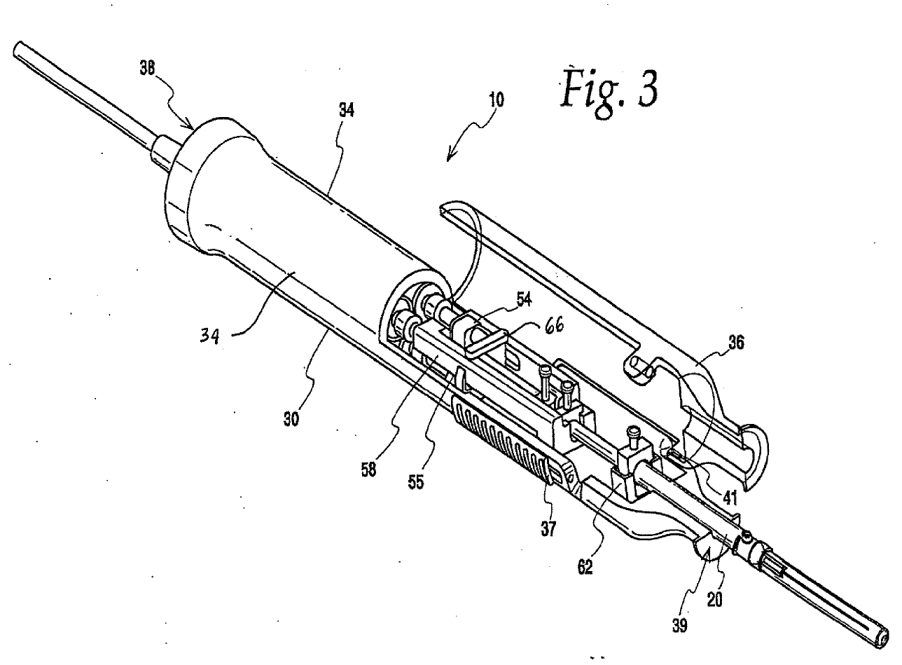

One example of an implantation apparatus 10 and system embodying the present invention is shown in FIGS. 3-9. Although apparatus 10 shown in FIGS. 3-9 is preferably a handheld type implantation apparatus where relative movement of the needle, optional guidewire and plunger is accomplished automatically by pre-programmed instructions in a microprocessor-based controller and at least some of the steps may be manually performed by the surgeon, apparatus 10 can also be used in a fully automated environment. In any event, implantation apparatus 10 shown in FIG. 3 includes a reusable portion 30 and a disposable portion embodied in needle assembly 20. As will be discussed in greater detail below, needle assembly 20 is separately provided and is received by arm sub-assembly 55 of implantation apparatus 30.

As shown in FIG. 3, implantation apparatus 10 includes a generally cylindrical body or housing 34, although as will be appreciated from other embodiments disclosed herein, the body shape of housing 34 is not critical. However, if apparatus 10 is to be held by the surgeon (i.e., a handheld apparatus) the shape of housing 34 should be such that is ergonomical, allowing for comfortable grasping by the surgeon. Housing 34 is closed at its proximal end by end cap 38 and has an opening 39 at its distal end through which at least a portion of needle assembly 20 extends. Door 36 provides access to the interior of housing 34 allowing for easy insertion and removal of needle assembly 20. Locking means such as slide lock 37 may be provided to secure door 36 to (and release door 36 from) housing 34. Door 36 may be secured to housing 34 by a hinge 41 allowing the door to swing open when it is unlocked. In an alternative embodiment, door 36 may be slidably attached to housing 34 and access to the interior of housing 34 may be achieved by sliding door 36 toward the proximal end of the housing 34. Of course, it will be appreciated that other ways of providing access to the interior of the implantation apparatus 10 are also possible.

Housing 34 and door 36 may be made of any material that is suitable for use in medical devices. For example, housing 34 may be made of a lightweight aluminum or, more preferably, a biocompatible plastic material. Examples of such suitable plastic materials include polycarbonate and other polymeric resins such as DELRIN (polymeric resin) and ULTEM (polymeric resin). Similarly, door 36 may be made of a plastic material such as the above-described materials including polymers and polymer resins such as polycarbonate, DELRIN (polymeric resin) and ULTEM (polymeric resin). In a preferred embodiment, door may be substantially translucent or transparent.

Re-usable portion 30 of implantation apparatus 10 houses the components required to effect movement of the needle assembly 20 components during the implantation procedure. As shown in FIGS. 3-6, implantation apparatus 10 houses a plurality of moveable arms, collectively referred to herein as the arm sub-assembly 55, which is adapted to receive needle assembly 20. Arms 54, 58 and 62 are axially moveable between the proximal and distal ends of apparatus 10 and are coupled to lead screws 52(a)-(c) at their distal ends which, in turn, are coupled to one or more drivers 44, 46, 48. In the embodiment shown in FIGS. 3-6, drivers are preferably a plurality of gear or stepper motors 44, 46 and 48. Alternatively, arms may be driven pneumatically or otherwise.

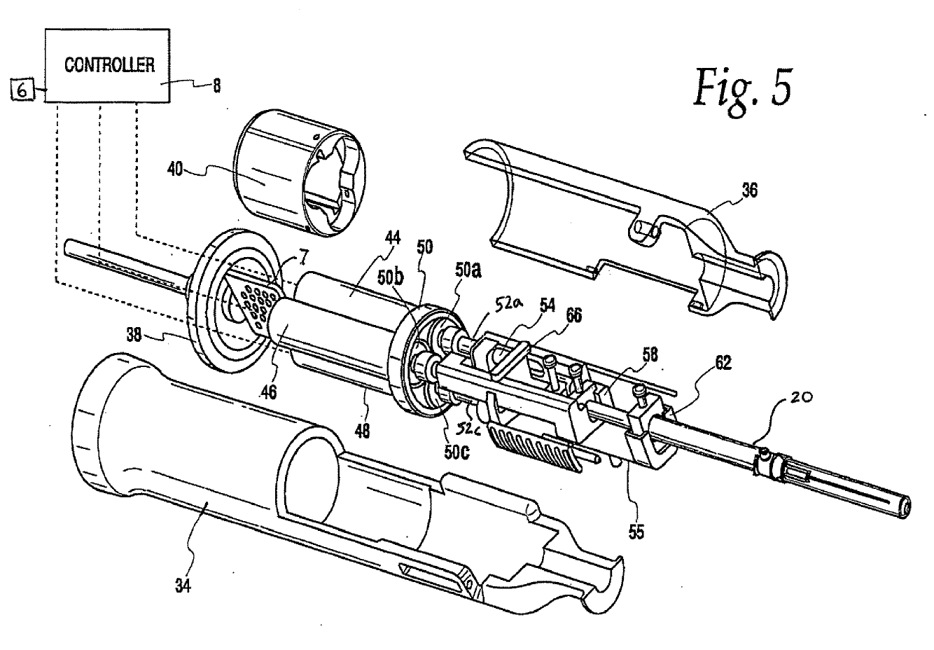

With respect to the embodiments of FIGS. 3-6, motors 44, 46 and 48 are housed near the proximal end of implantation apparatus 10. Motors 44, 46 and 48 may be stacked or bundled in parallel in the manner shown in FIG. 5 and held in place by front motor mount 50 and rear motor mount 40.

As indicated above, each of the motors 44, 46 and 48 (or other drivers) is coupled to one of the lead screws 52(a)-(c), which, in turn, are coupled to movable arms 54, 58 and 62 of arm sub-assembly 55. For example, with specific reference to the embodiment of FIGS. 3-6, lead screw 50(a) is coupled to guidewire arm 54; lead screw 50(b) is coupled to plunger arm 58; and lead screw 50(c) is coupled to needle arm 62. Motors 44, 46 and 48 may be selectively and independently activated by switches on the apparatus 10 itself or as schematically shown in FIG. 5 as described, may be coupled to a remote controller 8 of the system. In one embodiment, apparatus 10 includes printed circuit board 7 which establishes an electrical connection between motors 44, 46 and 48 and controller 8. Controller 8 may include a control box that supplies power and pre-programmed positioning instructions to the implantation apparatus 30 generally and motors 44, 46 and 48, specifically. Movements of the various arms 54, 58 and 62 can be initiated by the surgeon via a foot switch or other type of remote control 6.

As shown in the Figures, arms 54, 58 and 62 are preferably of varying axial lengths. Each of the arms 54, 58 and 62 includes a slot for receiving a portion of the needle assembly 20 (described below.) Thus, guidewire arm 54 includes a guidewire hub slot 57; plunger arm 58 includes a plunger hub slot 59 and needle arm 62 includes a needle hub slot 63.

In a preferred embodiment, each of the arms 54, 58 and 62 includes at its distal and/or proximal ends a portion having an enlarged cross-section. The distal “blocks” 54(a), 58(a) and 62(a) provide abutment surfaces which limit axial movement of the respective arms. As will be seen from the discussion of the implantation method, the distal blocks which also define slots 59, 62 and 63 limit movement of the particular arms, thereby ensuring that the guidewire, plunger and shunt 26 do not move beyond a pre-determined distance. Similarly, wall 65 of housing 34 limits movement of needle arm 62, likewise ensuring that the needle does not penetrate the eye beyond a desired distance. Proximal blocks 58(a), 58(b) and 58(c) (not shown) likewise provide an abutment surfaces for contacting fixed collars 53 on lead screws 52(a)-(c). Contact between the surfaces of blocks 58(a), 58(b) and 58(c) and respective collars 53 provides an indication that arms of arm subassembly 55 are in their rearmost or “hard stop” position, discussed below. Blocks 58(a)-(c) also include internal threaded nuts through which lead screws 50(a)-(c) travel.

As further seen in FIGS. 3-6, implantation apparatus 10 includes a guide block 66 attached to needle arm 62. Guide block 66 defines two partially enclosed apertures for slidably retaining guidewire arm 54 and plunger arm 58. Guide block 66 prevents rotation or other undesired dislocation of guidewire arm 54 and plunger arm 58 and maintains these components in an axially aligned orientation. Guide block 66 also serves as a stop that limits movement of arms 54 and 58.

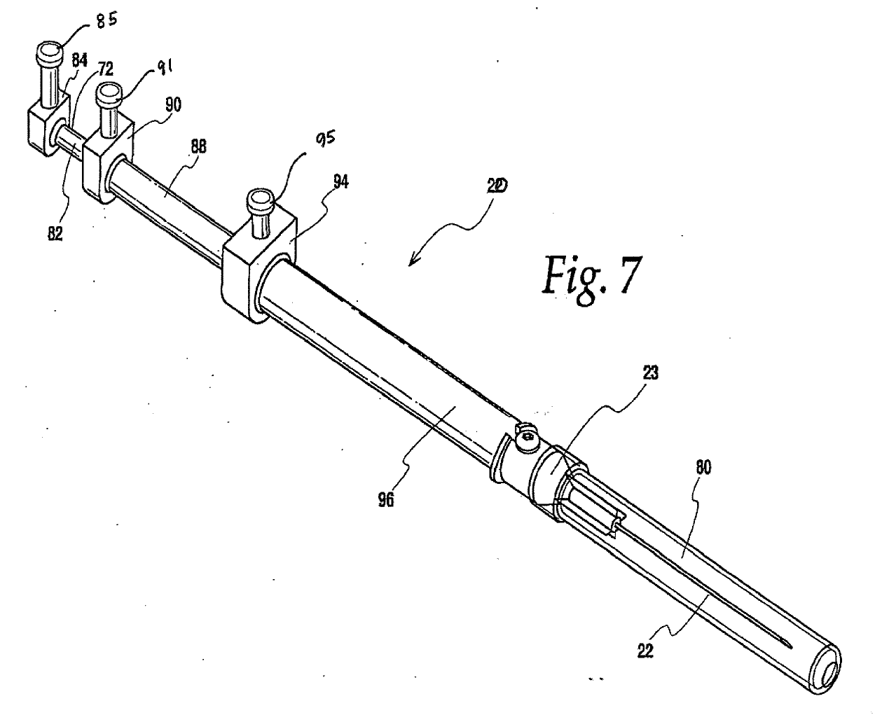

As noted above, arm sub-assembly 55 is adapted to receive needle assembly 20. Needle assembly, shown in FIGS. 7 and 8 is itself made of a plurality of separate, and co-axially assembled parts. Co-axial assembly of these constituent parts allows for relative axial movement of optional guidewire 28, needle 20 and plunger 32. As shown in FIGS. 7 and 8, in one embodiment, needle assembly includes a guidewire hub 72. In the embodiment shown, guidewire hub 72 includes a distal cylinder 82 and a proximal block 84. Guidewire 28 extends from the cylinder 82 and is received within plunger hub 68 which likewise includes a distal hollow cylinder and proximal block 90. Plunger tube 32 extends from plunger cylinder 88 and when brought together with guidewire hub 72 surrounds guidewire 86 along most of its length. Both guidewire 28 and plunger tube 92 are then received by needle hub 96. A hollow needle 22 attached to needle mount 23 is mounted on needle hub 96. Hollow needle 22 has an inner diameter sufficient to receive the assembled co-axial guidewire 86 and plunger 92. Of course, it will be appreciated that in certain embodiments, a guidewire may not be required and that needle assembly 20 may include a plunger and needle only.

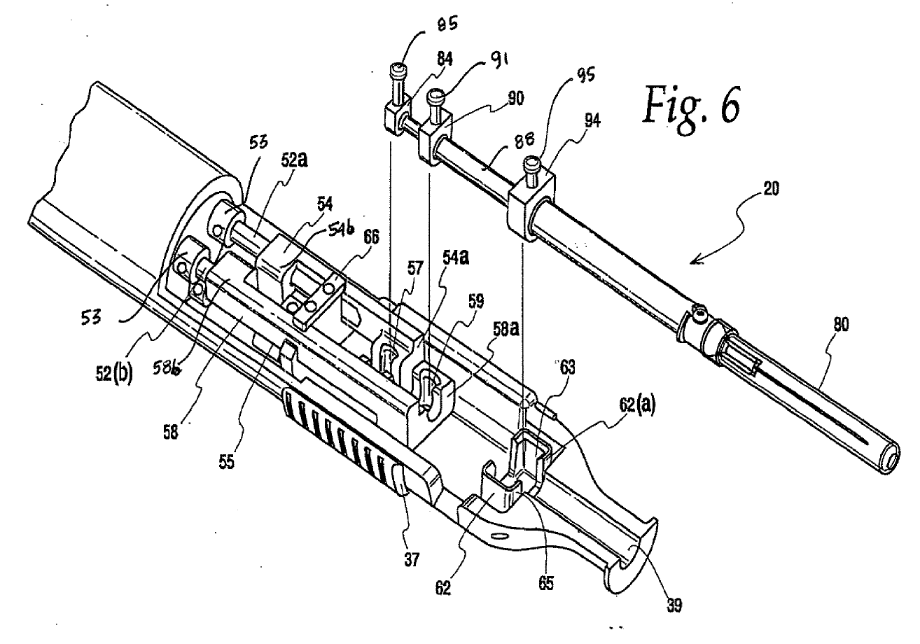

As best shown in FIG. 6, needle assembly 20 is adapted for placement within arm assembly 55. More specifically, guidewire block 84, plunger block 90 and needle block 94 of needle assembly 20 are received by the slots 57, 59 and 63, respectively, of arm sub-assembly 55. Each of blocks 84, 90 and 94 may include an upstanding pin 85, 91 and 95 (respectively). Pins 85, 91 and 95 are of a height sufficient so as to almost contact the inner surface of door 36 (when closed). Providing pins of sufficient height keeps needle assembly from becoming dislodged from sub-assembly 55 in the event that apparatus 10 is rotated by the surgeon. As shown in FIGS. 7 and 8, hollow needle 22 is preferably protected prior to use by removable needle cap 80.

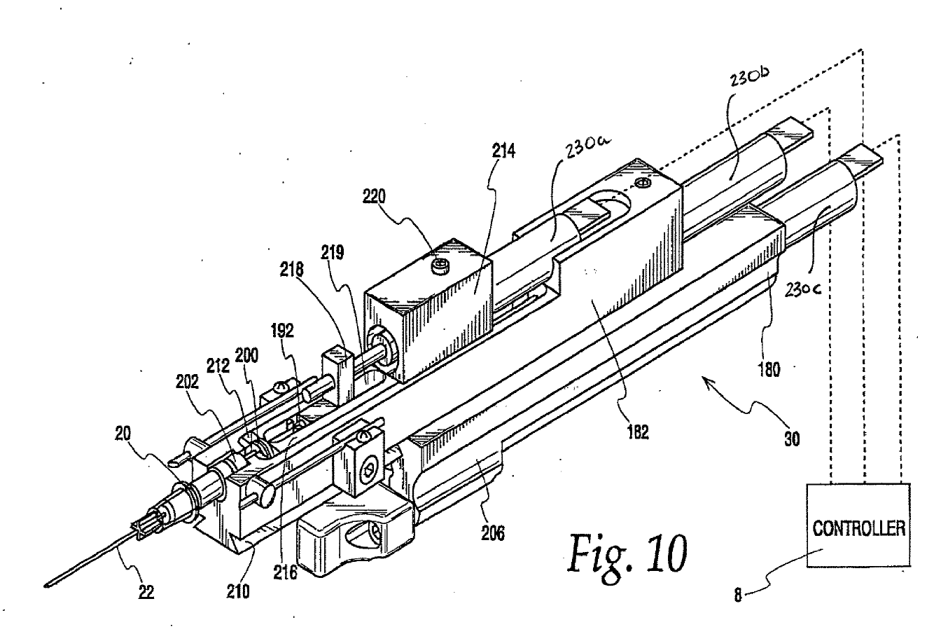

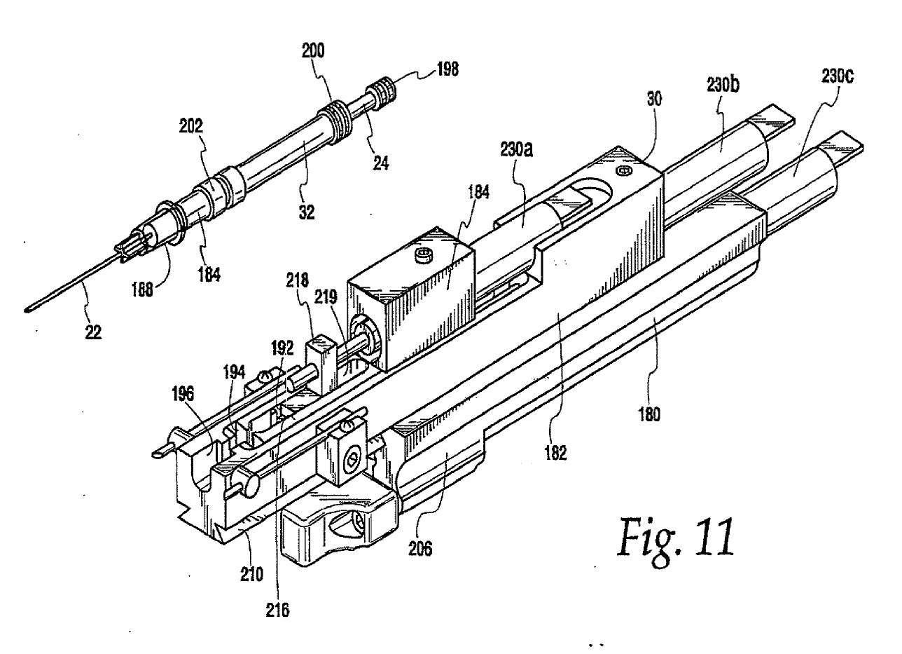

Another embodiment of a handheld implantation apparatus is shown in FIGS. 10-11. As in the embodiment described above, hand-held implantation apparatus 10 of FIG. 10 includes a reusable portion 30 that includes handle 180, movable block 182 and slider assembly 214. As with the embodiment of FIGS. 3-9 above, needle assembly 20, itself includes several different components that can be preassembled (as shown in FIG. 10) and are axially movable relative to one another. For example, in the embodiment shown in FIG. 10, needle assembly 20 includes plunger 32, needle adapter 184 and guidewire holder 24. Plunger 32 has a hollow cylindrical body which has an open distal end and an open proximal end. Open proximal end of plunger 32 receives guidewire 28 and guidewire holder 24.

As further shown in FIG. 10, distal end of plunger 32 is received by hollow needle adapter 184 and needle adapter 184 receives disposable needle 22. Needle 22 includes a distal piercing end and a hub 188 which is fitted over needle adapter 184. Once assembled, guidewire extends from guidewire holder 24 through plunger 32, through needle adapter 184 and needle 22. In the embodiments of FIG. 10, shunt 26 is typically placed on guidewire 28 near the distal end thereof within hollow needle 22.

Needle assembly 20 is mounted onto reusable handheld portion 30. More particularly, as shown in FIG. 3, needle assembly is fitted into slots 192, 194 and 196 of implantation apparatus 30. For example, collar 198 of guidewire holder 24 is received within slot 192, collar 200 of intermediate tube 32 is positioned within slot 194, and collar 202 of needle adapter 34 is received within slot 196.

Implantation apparatus 10 includes a handle 180. Handle 180 preferably includes groove 206 along the side wall for easy gripping by the surgeon. As shown in FIGS. 10 and 11, handle 204 supports movable slider block 182. Block 182 includes a slide 210 that fits within a central slot of handle 180. During use of implantation apparatus 10, block 182 may move axially within the slot of handle 180. Movable slider block 182 may also include a slot 212 (see FIG. 10) which receives plunger block assembly 214. As shown in the figures, plunger block 214 may be slidable within block 182. Plunger block assembly 214 includes forwardly extending arms 216 which defines at its distal end a slot 192 (in which collar 25 of guidewire holder 24 is received). Plunger block assembly also includes guidewire slider block 218 that is movable within slot 219 defined by arms 216. Guidewire slider block 218 is coupled to motor 230 (discussed below) by screw 220.

Reusable portion 30 of handheld implantation apparatus 10 generally depicted in FIGS. 10 and 11 may further include drivers for selectively actuating movement of the component parts of needle assembly 20, such as needle 22, guidewire 28, plunger 32, and shunt 26. As in the embodiment of FIGS. 3-9, in the embodiment of FIGS. 10 and 11, the drivers for selectively moving these and other components may be one or more motors, such as gear or stepper motors. Motors 230 may be selectively activated to move the desired component of apparatus 10. In one non-limiting example shown in FIGS. 10 and 11, a plurality of stepper motors 230(a), (b) and (c) are carried by handheld implantation apparatus. Motors 230(a)-(c) may be selectively activated by switches on the apparatus itself, remote hand-operated switches, a foot-operated controller and/or an automatically controlled via a preprogrammed controller (i.e., computer) 8.

Regardless of the means of control, in the example shown in FIGS. 10 and 11, motor 230(a) causes movement of guidewire slider block 218. Movement of guidewire slider block 218 which holds collar 25 of guidewire holder 24 results in selective back and forth movement of guidewire 28. Motor 230(b) moves arm 216 within slot 212 which holds collar 200 of plunger 32, allowing for back and forth movement of plunger 32. Finally, motor 230(c) drives block 182 including the entire needle assembly 20 further including block 214 and its associated components.

Of course, as described in relation to the embodiment of FIGS. 10-11, means for advancing or moving the operative components of handheld implantation apparatus 30 of FIGS. 11-12 need not be electrical and/or motor driven. Other embodiments of a handheld apparatus 10 that include other ways for actuating movement of the individual components may also be employed. For example, as shown in FIGS. 12-15, in alternative embodiments of a handheld implantation apparatus, the apparatus 10 may include mechanical means for selectively advancing the component parts of the needle assembly and the handheld implantation apparatus.

Turning to FIG. 12, implantation apparatus 110 includes a reusable handheld portion 112 that receives a disposable needle assembly 114. Implantation apparatus 110 includes a thumbwheel 116 placed on and movable along threaded screw 118. Attached to thumbwheel 116 is a syringe body 120. Distal end of syringe 120 receives needle assembly 122. Implantation apparatus 110 includes a conduit that extends through the handle 113 and is adapted for receiving guidewire 28.

Placement of shunt 26 onto guidewire 28 may be achieved by turning thumbwheel 116 in a first direction to retract needle assembly 122 and hollow needle 124, thereby revealing the distal end of guidewire 28 and plunger tube 32. At that point, shunt 26 is placed (typically manually) on guidewire 28 so that the proximal end thereof (the end opposite the leading end of shunt 26) of shunt comes into contact with the distal end of plunger 32. Thumbwheel 116 is then turned in an opposite direction to the first direction to slide needle 124 over plunger tube 32 and shunt 26.

Shunt 26 is now ready for implantation. During the implantation process, needle 124 is inserted into the eye and, more specifically, the cornea 19 or surgical limbus 17 of the eye in the manner described above and in U.S. Pat. No. 6,544,249. Needle 124 is advanced across anterior chamber 16 and into the sub-conjunctival space 18, stopping short of the conjunctiva 14. Thumbwheel 116 is then rotated again in the first direction to retract needle 124 and thereby expose shunt 26. Once in place, guidewire is retracted, releasing microfistula 26 from guidewire 28. Retraction of guidewire may be achieved manually by a simple pulling of guidewire 28 at the proximal end of apparatus 110. Once shunt 26 is in its final position, needle 124 is removed.

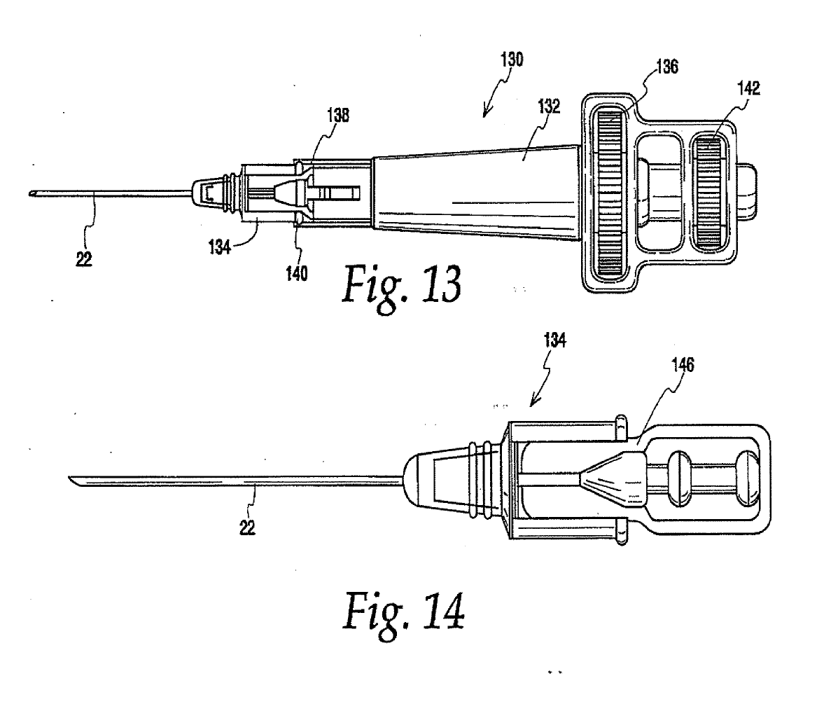

FIGS. 13 and 14 illustrate another embodiment of a handheld implantation apparatus 130 that likewise utilizes mechanical means for advancing and/or selectively moving the component parts of the needle assembly and/or apparatus 130. As in the embodiment of FIG. 12, handheld implantation apparatus relies on mechanically driving the component parts. As shown in FIG. 13, implantation apparatus 130 includes handle portion 132 with a needle assembly 134 attached to the distal end of body 132. A thumbwheel 136 is rotatable and coupled to an internal screw (not shown). Internal screw is attached to arms 138 which grasp flange 140 of needle assembly 134, such that turning of thumbscrew 136 effects axial movement of needle assembly 134.

In contrast to the embodiment of FIG. 12, implantation apparatus 130 may further include additional means for controlling movement of other components of the implantation apparatus. For example, in the embodiment of FIG. 13, a second thumbwheel 142 is mechanically coupled to guidewire 28. A rotation of thumbwheel 142 allows for retraction of guidewire 28 after implantation of shunt 28.

FIG. 14 provides an enlarged view of needle assembly 134 shown in FIG. 13. As seen in FIG. 14, an assembly retainer 146 is provided. Assembly retainer 146 is affixed to the needle assembly 134 during shipment to prevent movement of guidewire 28 and control tube. Retainer is removed prior to insertion of the needle assembly 134 onto the handle 132 of apparatus 130.

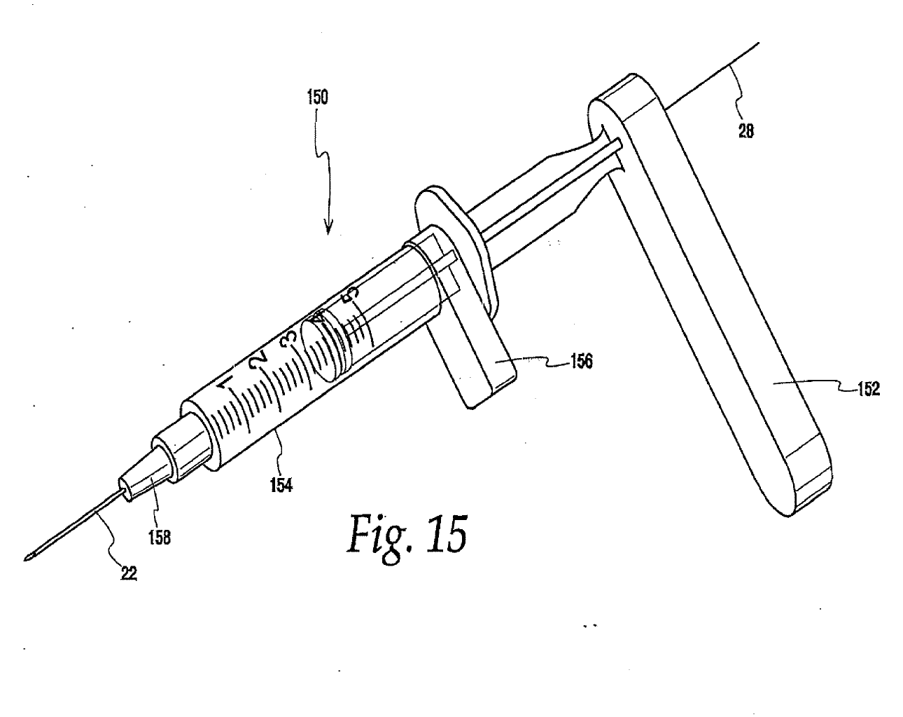

FIG. 15 shows another embodiment of an implantation apparatus. The implantation apparatus 150 of FIG. 8 includes a handle 152, a movable or slidable syringe portion 154 and a trigger 156 for actuating movement of slidable syringe 124. Implantation apparatus 150 further includes an attachable needle assembly 158 (with needle 22) at the distal end of syringe 154. As shown in FIG. 15, guidewire 28 extends through implantation apparatus 150 in similar fashion to the apparatus of FIG. 12. Guidewire 28 extends through barrel 154 and carries a tube 32 near its distal end. Barrel 154 is preferably filled with gas (e.g., air, CO.sub.2, nitrogen or liquid (e.g., water, trypan blue, saline or a viscoelastic solution).

For placement of shunt 26 onto guidewire 28, trigger 156 is pulled, resulting in rearward movement of syringe 154 and needle 22. Rearward movement of needle 22 exposes guidewire 28 and allows for placement of shunt 26 onto guidewire. Release of the trigger 158 advances needle 22 to cover guidewire 28 and shunt 26. As in the previous embodiments, needle 22 pierces cornea 19 or surgical limbus 17, and is advanced through anterior chamber 16 to the desired location of the eye (i.e. the area between the sub-conjunctival space 18 and the anterior chamber). Trigger 156 is once again pulled to move needle assembly 158 in a rearward direction thereby exposing shunt 26 carried by guidewire 28. Once the surgeon has determined that the shunt 26 is in the desired location, guidewire 28 is retracted, thereby releasing shunt 26. As shown in FIG. 15, retraction of guidewire 28 may be performed manually, as in the embodiment of FIG. 12, by simply pulling guidewire 28. Alternatively, mechanical means for moving guidewire, as in the examples of FIGS. 12 and 13, may also be provided.

Although selective movement of guidewire 28, needle assembly, plunger 32 or guidewire holder 24 with the shunt 26 using electrical, mechanical or even some manual means have been described, other means for actuating movement of these components may also be used instead of or in addition to such means. For example, movement of the various component parts may be achieved by pneumatic control or fluidic control.

The method of implanting shunt 26 using implantation apparatus will now be described. The method will be described with particular reference to the embodiment of FIGS. 3-9, although many of the steps described may also be employed using other embodiments of the implantation apparatus. In addition, depending on the type of apparatus and type of shunt used, there may be variations to some of the method steps. For example, in some embodiments, a guidewire may be omitted. In addition, the advancement and retraction steps of the parts of needle assembly may be continuous or incremental. Regardless of the apparatus used, the sequence of steps, distances traveled and continuous or incremental movement, the ultimate location of shunt 26 is substantially the same using any of the methods, systems and apparatus described herein.

At the outset, it will be appreciated that the implantation of shunt 26 requires precise placement of the shunt 26 in the correct location within the eye. Moreover, it will also be appreciated that the distances traveled by the shunt 26, plunger 32, guidewire 28 and needle 22 are typically measured in millimeters. Such precision may be difficult for even the most skilled surgeon to achieve by manual manipulation (due to natural hand tremors in humans). Accordingly, in embodiments other than the manual hand-held implanters in FIGS. 12-15, many of the actual implantation steps are preferably carried out under the automatic control of an external, preprogrammed controller 8. While the initial eye entry steps and some repositioning steps may be performed manually by the surgeon, steps related to the release and location of shunt 26 may be automatically controlled.

In a first step, preferably performed during factory assembly, shunt 26 is loaded into needle assembly 20. During loading, the distal tip of guidewire preferably extends slightly beyond the beveled tip of hollow needle 22. Shunt 26 may be manually placed on guidewire 28 until proximal end of shunt 26 contacts the distal end of plunger 32. Guidewire 28, with shunt 26 placed thereon is then retracted into hollow needle 22.

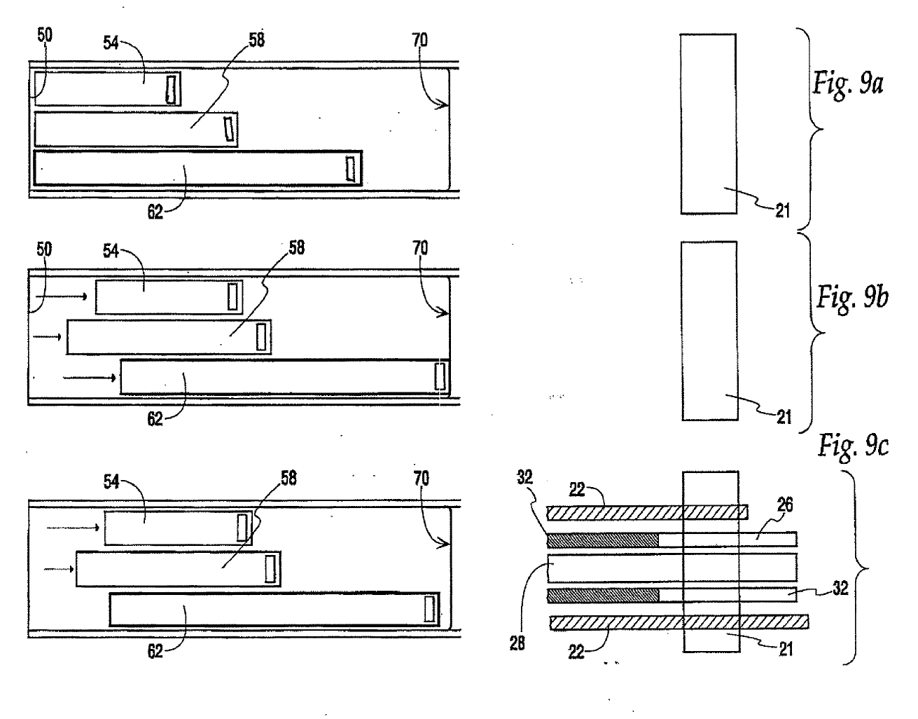

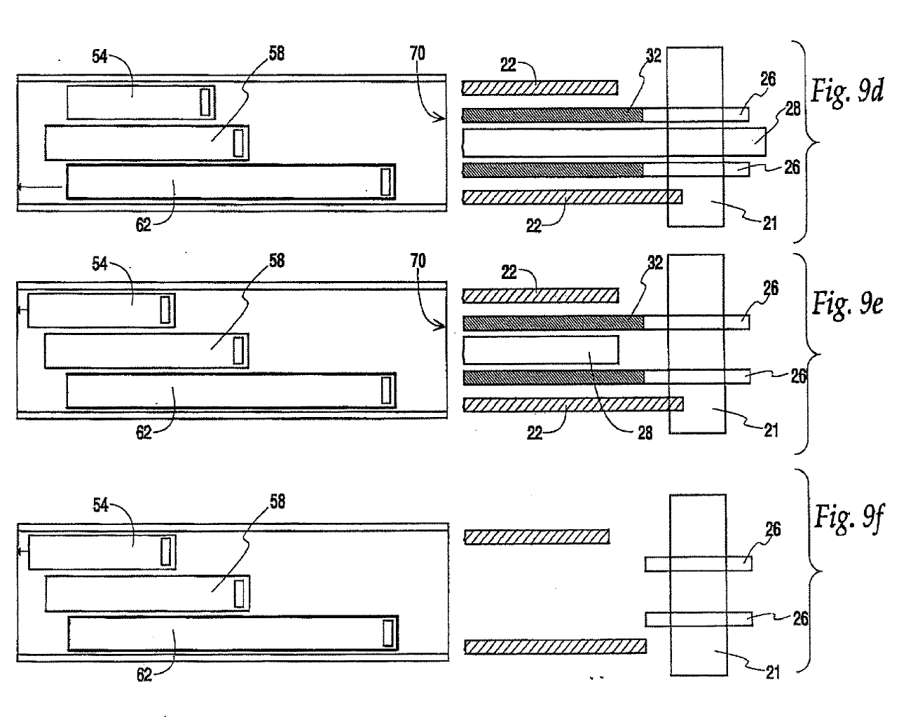

Prior to loading needle assembly 20 into apparatus 30, pre-positioning of arm-subassembly may be desired or required. Thus, in a first step, all motors are activated to retract guidewire arm 54, plunger arm 58 and needle arm 62 to a proximal most position such that the proximal end surfaces of the arms abut against collars 53. This “hard stop” position is shown schematically in FIG. 9a. The operator may then prepare implantation apparatus 30 for loading of needle assembly by activating each motor and advancing each arm assembly 55 to a “home” position and shown in FIG. 9(b). As will be seen in FIG. 9(b) movement of needle arm 62 is restricted by wall 70 of apparatus 30. With the motors properly aligned in the “home” position, needle assembly is installed by inserting guidewire hub block 86 into guidewire hub slot 57; plunger hub block 90 into plunger hub slot 59 and needle hub block into slot 63. With needle assembly 20 properly installed, the surgeon may begin the procedure by inserting the end distal tip of hollow needle 22 into the eye. As shown in FIG. 1 and as previously described, the surgeon inserts the hollow needle 22 into the anterior chamber via the cornea or surgical limbus of the eye and advances it either manually (or under automatic control) to a location short of the final implantation site. Alternatively, the surgeon may first make an incision in the eye and insert needle 22 through the incision. Once the needle 22 has been properly inserted and placed, the program may be activated to commence automatic implantation of shunt 26. In a first implantation step, simultaneously motors) 44 and 46 are activated to advance guidewire arm 54 and plunger are 58 as shown in FIG. 9(c) which thereby advances shunt 26 forward into the subconjunctival space of the eye, as generally depicted in FIG. 9(c). For example, in one embodiment, plunger 32 and guidewire 28 are advanced approximately a total of 2 millimeters. Preferably, the rate of placement of shunt is carefully controlled because it allows the shunt to absorb fluid from the surrounding tissue thereby causing it to swell and to provide better anchoring in the tissue. Rapid advancement or placement of microfistula shunt 26 may not allow tube 26 to adequately swell which can possibly result in unwanted migration of shunt 26 after implantation. In one embodiment, the rate of placement may be between approximately 0.25-0.65 mm/sec.

After the advancement of the plunger and guidewire described above, motor 48 is activated and needle arm 62 is moved in a rearward direction such that needle 22 is withdrawn from its position shown in FIG. 9(c) to the position shown in FIG. 9(d). Withdrawal of needle 22 should preferably expose the entire length of shunt 26, and, in addition, the distal end of the plunger, thereby allowing the surgeon to visualize the final position of the proximal edge of the shunt. In one embodiment, the distance that hollow needle 22 is withdrawn is approximately 4.2 millimeters. At this point, the program prompts (e.g., audibly) the surgeon to visually view the location of shunt 26 and determine if it is correctly placed. The surgeon can manually make any adjustments to a desired position by moving the implanter forward or backward. The automatic system may be programmed to allow the surgeon sufficient time to make any further manual adjustments and may require the surgeon to press the foot or other switch or otherwise effect movement to continue delivery of the shunt. After a selected period of time, the automated program preferably resumes control of implantation procedure by activating motor guidewire motor 44, to retract guidewire arm 54 and thus withdraw guidewire 28 as shown in FIG. 9(e). Removal of the guidewire preferably occurs in one single step as shown in FIG. 9(e). Finally, the system will then preferably alert the surgeon that the procedure is now complete and the needle 22 may be withdrawn (manually or automatically) from the eye as shown in FIG. 9(f).

From the preceding discussion, it will be appreciated that bioabsorbable microfistula shunt is implanted by directing the needle across the anterior chamber, entering the trabecular meshwork (preferably between Schwalbe's Line and the Scleral spur), and directing the needle through the sclera until the distal tip of the needle is visible in the subconjunctival space. The length of the shunt through the sclera should be approximately 2-4 mm. Once the surgeon has placed the needle in this location, he may actuate the implanter to begin the release steps. The shunt is released and the needle is withdrawn such that approximately 1-2 mm of the shunt resides in the sub conjunctival space, approximately 2-4 mm resides in the scleral shunt, and approximately 1-2 mm resides in the anterior chamber. Once the shunt is released, the surgeon removes apparatus needle 20.

Proper positioning of the bioabsorbable shunt 26 should be carefully controlled for at least the following reasons. If the surgical procedure results in the formation of a bleb, the more posterior the bleb is located, the fewer complications can be expected. Additionally, the bleb interferes less with eyelid motion and is generally more comfortable for the patient. Second, a longer scleral shunt provides more surface contact between the shunt and the tissue providing better anchoring. Third, the location of the shunt may play a role in stimulating the formation of active drainage structures such as veins or lymph vessels. Finally, the location of the shunt should be such so as to avoid other anatomical structures such as the ciliary body, iris, and cornea. Trauma to these structures could cause bleeding and other complications for the patient. Additionally, if the bleb is shallow in height and diffuse in surface area, it provides better drainage and less mechanical interference with the patient's eye. Tall, anteriorly located blebs are more susceptible to complications such as conjunctival erosions or blebitis which require further intervention by the surgeon.

The ab interno approach provides better placement than the ab externo approach because it provides the surgeon better visibility for entering the eye. If directing the needle from an ab externo approach, it is often very difficult for the surgeon to direct the needle to the trabecular meshwork (between Schwalbe's line and the scleral spur) without damaging the cornea, iris, or ciliary body.

In an alternative method of implantation, it is possible to direct the needle from the trabecular meshwork into the suprachoroidal space (instead of the subconjunctival space) and provide pressure relief by connecting these two spaces. The suprachoroidal space also called supracilliary space has been shown to be at a pressure of a few mmHg below the pressure in the anterior chamber.

Common to all of the embodiments of handheld implantation apparatus are a needle assembly including a hollow needle. In a preferred embodiment, hollow needle 22 may be any needle suitable for use in medical procedures. As such, needle 22 is made of a hard and rigid material such as stainless steel with a beveled sharpened distal tip. Needle 22 is bonded, welded, overmolded, or otherwise attached to the needle mount 23 and/or hub that is adapted for placement onto the distal end of a needle assembly. The needle 22 is disposable and intended for one time use.

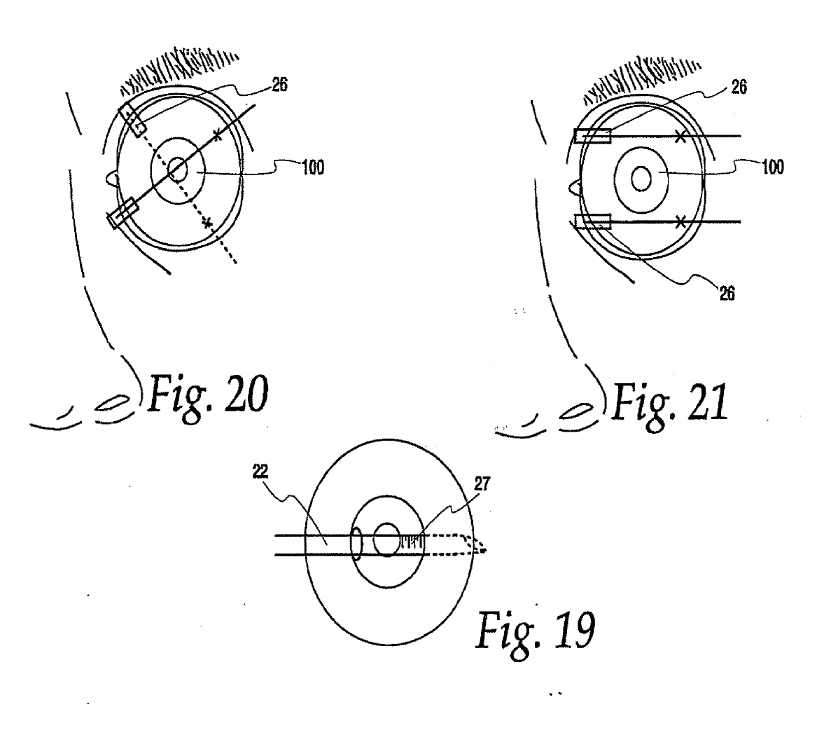

Hollow needle 22 and indeed, the entire needle assembly may be sterilized by known sterilization techniques such as autoclaving, ethelyne oxide, plasma, electron beam, or gamma radiation sterilization. In a preferred embodiment, needle 22 is a 25 gauge thin walled needle that is commercially available from Terumo Medical Corp., Elkton, Md. 21921. The inside diameter of hollow needle 22 must be sufficient to accommodate optional guidewire 28, shunt 26 and plunger tube 32, with an inner diameter of 200-400 um being preferred. The usable length of needle 22 may be anywhere between 20-30 mm, although a length of approximately 22 mm is typical and preferred. Preferably, needle 22 may include markings or graduations 27 near the distal tip as shown in FIG. 19. A graduated needle may be particularly useful to a surgeon inasmuch as much of the needle within the eye is not visible to the surgeon. Typically, the only visible portion of needle 22 is the portion within the anterior chamber. Accordingly, graduations 27 uniformly spaced along the needle shaft assist the surgeon in determining how far to advance the needle in order to place shunt 26 in the desired location. In one embodiment, the graduations may be applied using laser marks, ink, paint or engraving and are typically spaced 0.1 to 1.0 mm apart.

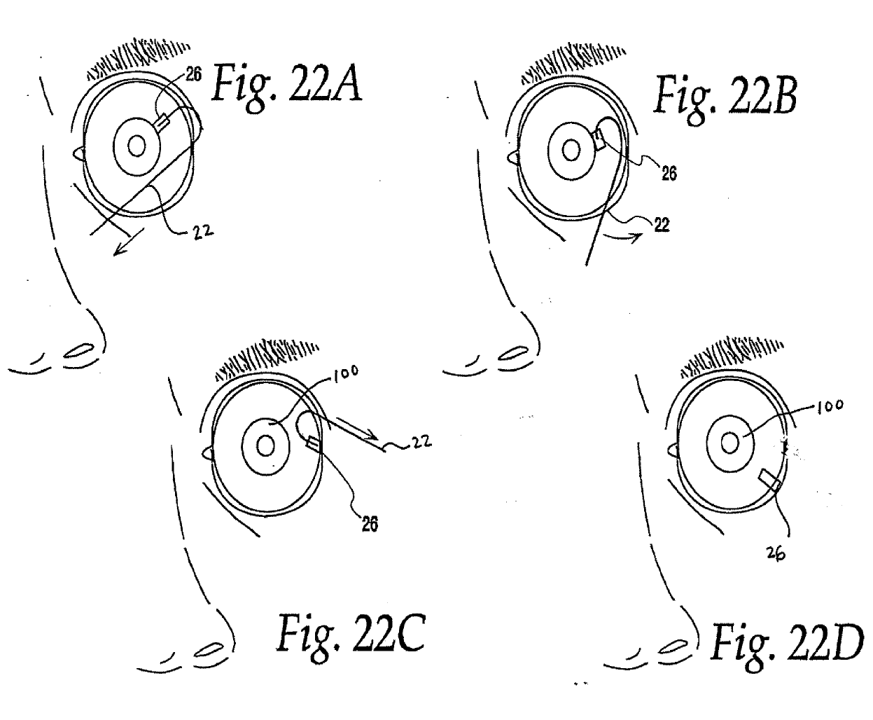

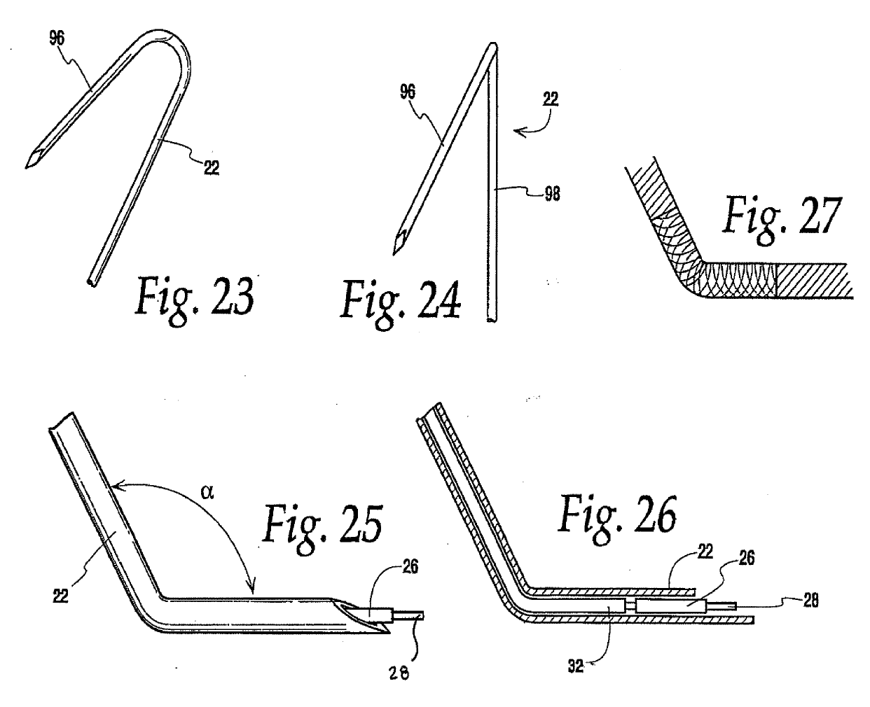

While a straight hollow needle of the type typically used in medial procedures is generally preferred, in an alternative to the needle shown in the FIGS. 3-15 and described above, needle 22 may be rigid and have a distal portion that is arcute as shown in FIGS. 22-24. As shown in FIGS. 22(a)-(d) and FIGS. 23-24 arcuate needle may be preferably U-shaped or substantially U-shaped. With an “arcuate” needle, instead of pushing the needle into the patient's eye, the surgeon may orient the needle to “pull” the needle into the patient's eye. As shown in FIGS. 23-24, the distal portion of the needle 22 terminating in the beveled tip, identified by reference number 96 is preferably disposed obliquely relative to the longitudinal axis of needle shaft 98 as seen in FIG. 24.

Providing a piercing end 96 that is bent away from the plane of needle shaft 98 can facilitate manipulation and rotation of needle 22 during implantation of tube 26. It may also provide the surgeon with greater flexibility in terms of selecting the corneal entry site and the ultimate final position of shunt 26. This is perhaps best seen with reference to FIGS. 20, 21 and 22(a)-(d).

For example, FIG. 20 depicts a transpupil implantation delivery generally described in U.S. Pat. No. 6,544,249 as shown in FIG. 1. While the approach is satisfactory, it does require the needle to cross the visual axis. In the event of a surgical error that causes damage to the cornea or lens, corrective surgery may be required.

FIG. 21 depicts an alternative method of delivery referred to as an ipsilateral tangential delivery of shunt 26. In the ipsilateral tangential delivery method, the straight needle is directed tangentially to the pupil 100 border and the surgical limbus. This type of implant delivery allows the shunt to be delivered to a greater circumference of the eye and has the advantage of avoiding the visual axis. Avoiding the visual axis reduces the risk of complications to the cornea 19 and lens through contact during surgery. Ipsilateral tangential delivery is a modification of the transpupil implant location generally described in U.S. Pat. No. 6,544,249, previously incorporated by reference.

Although the transpupil implant delivery and/or the ipsilateral tangential delivery, if performed correctly, are acceptable methods of delivering shunt 26, they do somewhat limit the location of the corneal entry site due to interference with the nose and eye orbit bones. In that regard, an arcuate needle of the type described above and shown in FIGS. 22(a)-(d) and FIGS. 23-24 may provide greater flexibility to the surgeon. With an arcuate needle, shunt 26 may be placed anywhere around the 360° circumference of the eye, including the temporal quadrants which would not be otherwise accessible for the reasons discussed above.

A further advantage of the arcuate needle and the delivery implant method associated therewith is that microfistula shunt 26 can be delivered without crossing the lens i.e., visual axis, thereby reducing the risk of complications. An arcuate needle design may also allow the surgery to be done in patients with abnormal anatomy or who have previously undergone surgery.

In accordance with delivering a microfistula shunt 26 using the U-shaped hollow needle 20 of FIGS. 23 and 24, as noted above, instead of pushing the needle into the patient's eye, the surgeon orients the needle to “pull” needle 22 into the patient's eye. Thus, as shown in FIG. 22(a), the pointed tip of hollow needle 22 is inserted at the desired corneal entry point and pulled in the direction of the arrow. Once the portion of needle 22 that contains the shunt 26 is in the patient's eye, the surgeon rotates the needle and directs the needle 22 toward the target within the angle of the anterior chamber. After adjusting needle 22 to the proper position, the surgeon again pulls the needle 22 in the direction of the arrow of FIG. 22(b) so that the needle is directed through the trabecular mesh work and sclera. The particular advancement and delivery steps described previously are then performed to place the shunt 26 in the desired location and withdraw the guidewire plunger and needle from the eye. Of course, retraction and other movements of the needle may be automatically controlled in the manner described above and as shown in FIG. 9.

In a further embodiment, a hollow needle 22 that is bent (but not necessarily in a U-shape as described above), may be provided. A needle of this type is shown in FIGS. 25-27. As with the “arcuate” or U-shaped needles discussed above, a simple bend in the distal portion of needle 22 can likewise avoid interference from the patient's facial features. A bend that creates an angle .alpha. of between 90°-180° may be preferred. Providing a needle 22 with a bend is also ergonomically desirable in that it improves the position of the surgeon's hands during surgery. For example, by providing a bend in the distal portion of needle 22, a surgeon may rest and stabilize his hands on the patient's forehead or other support while making the initial corneal entry and carrying out the later implantation steps. Providing a bend in the distal portion of needle 22 is not merely an alternative to the U-shaped needle of FIGS. 23 and 24. In fact, both features i.e., a needle with an arcuate distal portion and further having a bend near the distal tip may be employed together in the needle 22.

Whether the needle is U-shaped or bent at an angle .alpha. shown in FIG. 25, the component parts of needle 22 must likewise be susceptible to bending. Accordingly, instead of a rigid plunger 32 and guidewire 28, both the plunger and guidewire may be, in part, bendable or be made of a material that is bendable, yet provides adequate support and has adequate strength. In one example, the plunger 32 may be made of a tightly wound coil such as but limited to a spring or coil. Alternatively, at least a portion of guidewire 28 or plunger 32 may be made of a flexible plastic material including a polymeric material, examples of which include polyimide, PEEK, Pebax or Teflon. Other bendable, flexible materials may also be used. Similarly, guidewire 28 may be made of any of the above-described materials or a material such as nitinol which has shape memory characteristics. The entire plunger or guidewire 28 may be made of the flexible materials described above or, as shown in FIG. 27 only a portion of the guidewire 28 or plunger tube 32 may be made of the selected material or be otherwise bendable.

Typically, however, guidewire 28 is preferably a narrow gauge wire made of a suitable rigid material. A preferred material is tungsten or stainless steel, although other non-metallic materials may also be used. In a preferred embodiment, guidewire 28 is solid with an outside diameter of approximately 50-200 (ideally 125) microns. Where guidewire 28 is made of tungsten, it may be coated with a Teflon, polymeric, or other plastic material to reduce friction and assist in movement of shunt 26 along guidewire 28 during implantation.

Shunts 26 useful in the present invention, are preferably made of a biocompatible and preferably bioabsorbable material. The materials preferably have a selected rigidity, a selected stiffness and a selected ability to swell (during manufacture and/or after implantation) in order to provide for secure implantation of the shunt in the desired section of the eye. Selecting a material that is capable of a controlled swelling is also desirable. By controlled swelling, it is meant that the swellable material is such that the outer diameter of the shunt expands (increases) without decreasing the inner diameter. The inner diameter may increase or remain substantially the same. The materials and methods for making shunts described below provide such controlled swelling. By sufficient biocompatibility, it is meant that the material selected should be one that avoids moderate to severe inflammatory or immune reactions or scarring in the eye. The bioabsorbability is such that the shunt is capable of being absorbed by the body after it has been implanted for a period of anywhere between 30 days and 2 years and, more preferably, several months such as 4-7 months.

In one embodiment, the material selected for the shunts is preferably a gelatin or other similar material. In a preferred embodiment, the gelatin used for making the shunt is known as gelatin Type B from bovine skin. A preferred gelatin is PB Leiner gelatin from bovine skin, Type B, 225 Bloom, USP. Another material that may be used in the making of the shunts is a gelatin Type A from porcine skin also available from Sigma Chemical. Such gelatin is available is available from Sigma Chemical Company of St. Louis, Mo. under Code G-9382. Still other suitable gelatins include bovine bone gelatin, porcine bone gelatin and human-derived gelatins. In addition to gelatins, microfistula shunt may be made of hydroxypropyl methycellulose (HPMC), collagen, polylactic acid, polylglycolic acid, hyaluronic acid and glycosaminoglycans.

In accordance with the present invention, gelatin shunts are preferably cross-linked. Cross-linking increases the inter- and intramolecular binding of the gelatin substrate. Any means for cross-linking the gelatin may be used. In a preferred embodiment, the formed gelatin shunts are treated with a solution of a cross-linking agent such as, but not limited to, glutaraldehyde. Other suitable compounds for cross-linking include 1-ethyl-3-[3-(dimethyamino)propyl]carbodiimide (EDC). Cross-linking by radiation, such as gamma or electron beam (e-beam) may be alternatively employed.

In one embodiment, the gelatin shunts are contacted with a solution of approximately 25% glutaraldehyde for a selected period of time. One suitable form of glutaraldehyde is a grade 1G5882 glutaraldehyde available from Sigma Aldridge Company of Germany, although other glutaraldehyde solutions may also be used. The pH of the glutaraldehyde solution should preferably be in the range of 7 to 7.8 and, more preferably, 7.35-7.44 and typically approximately 7.4.+−.0.01. If necessary, the pH may be adjusted by adding a suitable amount of a base such as sodium hydroxide as needed.

Shunts used in the present invention are generally cylindrically shaped having an outside cylindrical wall and, in one embodiment, a hollow interior. The shunts preferably have an inside diameter of approximately 50-250 microns and, more preferably, an inside diameter and us, a flow path diameter of approximately 150 to 230 microns. The outside diameter of the shunts may be approximately 80-300 with a minimum wall thickness of 30-70 microns for stiffness.

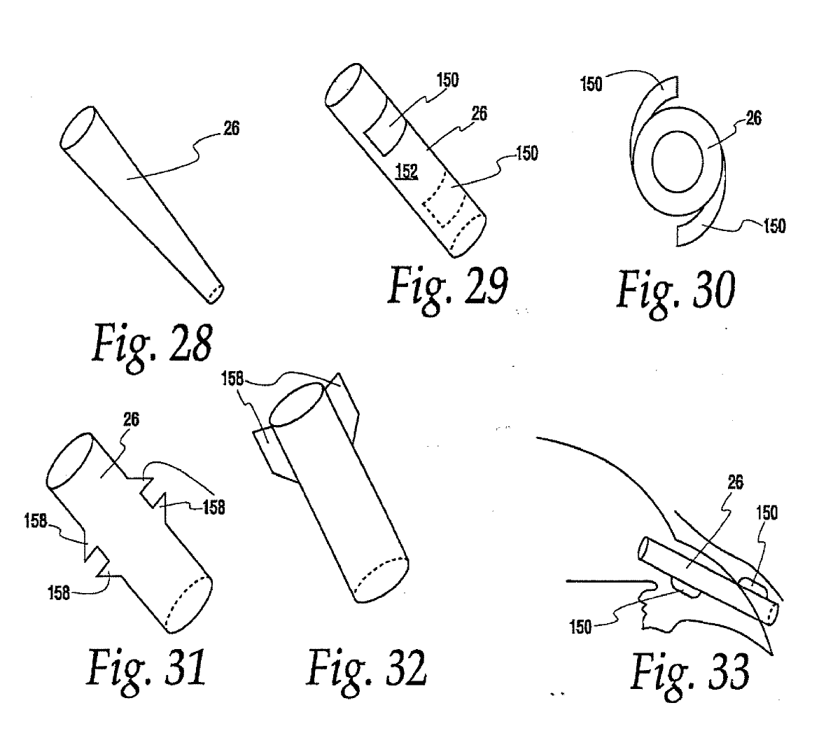

As shown in FIG. 28, one end of tube 26 may be slightly tapered to limit or prevent migration of tube 26 after it has been implanted. Other means for limiting migration are also shown in FIGS. 29-33. For example, shunt 26 may include expandable tab 150 along outer surface 152 of tube 26. As shown in FIG. 29, prior to deployment and introduction of tube into the patient's eye, tabs 150 are rolled or otherwise pressed against surface 152. Tabs 150 may also be features that are cut out of the outer surface of shunt 26 (i.e., not separately applied). Upon contact with an aqueous environment, tabs 150 are deployed. Specifically, contact with an aqueous environment causes tabs 150 to expand as shown in FIG. 30 and, thereby, create an obstruction which limits or prevents migration of tube 26. Tube 26 may include a plurality of tabs, typically but not limited to 1-4, and may be located nearer the subconjuctival side, the anterior chamber or both, as shown in FIG. 33. Other means for limiting or preventing migration include barbs 158 placed along the length of tube 26 as shown in FIGS. 31-32 and also disclosed in U.S. Pat. Nos. 6,544,249 and 6,007,511, previously incorporated by reference.

The length of the shunt may be any length sufficient to provide a passageway or canal between the anterior chamber and the subconjunctival space. Typically, the length of the shunt is between approximately 2 to 8 millimeters with a total length of approximately 6 millimeters, in most cases being preferred. The inner diameter and/or length of tube 26 can be varied in order to regulate the flow rate through shunt 26. A preferred flow rate is approximately 1-3 microliters per minute, with a flow rate of approximately 2 microliters being more preferred.

In one embodiment, shunts 26 may be made by dipping a core or substrate such as a wire of a suitable diameter in a solution of gelatin. The gelatin solution is typically prepared by dissolving a gelatin powder in de-ionized water or sterile water for injection and placing the dissolved gelatin in a water bath at a temperature of approximately 55° C. with thorough mixing to ensure complete dissolution of the gelatin. In one embodiment, the ratio of solid gelatin to water is approximately 10% to 50% gelatin by weight to 50% to 90% by weight of water. In an embodiment, the gelatin solution includes approximately 40% by weight, gelatin dissolved in water. The resulting gelatin solution preferably is devoid of any air bubbles and has a viscosity that is between approximately 200-500 cp and more preferably between approximately 260 and 410 cp (centipoise).

The gelatin solution may include biologics, pharmaceuticals or other chemicals selected to regulate the body's response to the implantation of shunt 26 and the subsequent healing process. Examples of suitable agents include anti-mitolic pharmaceuticals such as Mitomycin-C or 5-Fluorouracil, anti-VEGF (such as Lucintes, Macugen, Avastin, VEGF or steroids), anti-coagulants, anti-metabolites, angiogenesis inhibitors, or steroids. By including the biologics, pharmaceuticals or other chemicals in the liquid gelatin, the formed shunt will be impregnated with the biologics, pharmaceuticals or other chemicals.

Once the gelatin solution has been prepared, in accordance with the method described above, supporting structures such as wires having a selected diameter are dipped into the solution to form the gelatin shunts. Stainless steel wires coated with a biocompatible, lubricious material such as polytetrafluoroethylene (Teflon) are preferred.

Typically, the wires are gently lowered into a container of the gelatin solution and then slowly withdrawn. The rate of movement is selected to control the thickness of the coat. In addition, it is preferred that a the tube be removed at a constant rate in order to provide the desired coating. To ensure that the gelatin is spread evenly over the surface of the wire, in one embodiment, the wires may be rotated in a stream of cool air which helps to set the gelatin solution and affix film onto the wire. Dipping and withdrawing the wire supports may be repeated several times to further ensure even coating of the gelatin. Once the wires have been sufficiently coated with gelatin, the resulting gelatin films on the wire may be dried at room temperature for at least 1 hour, and more preferably, approximately 10 to 24 hours. Apparatus for forming gelatin tubes are described below.

Once dried, the formed microfistula gelatin shunts are treated with a cross-linking agent. In one embodiment, the formed microfistula gelatin films may be cross-linked by dipping the wire (with film thereon) into the 25% glutaraldehyde solution, at pH of approximately 7.0-7.8 and more preferably approximately 7.35-7.44 at room temperature for at least 4 hours and preferably between approximately 10 to 36 hours, depending on the degree of cross-linking desired. In one embodiment, formed shunt is contacted with a cross-linking agent such as gluteraldehyde for at least approximately 16 hours. Cross-linking can also be accelerated when it is performed a high temperatures. It is believed that the degree of cross-linking is proportional to the bioabsorption time of the shunt once implanted. In general, the more cross-linking, the longer the survival of the shunt in the body.

The residual glutaraldehyde or other cross-linking agent is removed from the formed shunts by soaking the tubes in a volume of sterile water for injection. The water may optionally be replaced at regular intervals, circulated or re-circulated to accelerate diffusion of the unbound glutaraldehyde from the tube. The tubes are washed for a period of a few hours to a period of a few months with the ideal time being 3-14 days. The now cross-linked gelatin tubes may then be dried (cured) at ambient temperature for a selected period of time. It has been observed that a drying period of approximately 48-96 hours and more typically 3 days (i.e., 72 hours) may be preferred for the formation of the cross-linked gelatin tubes.

Where a cross-linking agent is used, it may be desirable to include a quenching agent in the method of making shunt 26. Quenching agents remove unbound molecules of the cross-linking agent from the formed shunt 26. In certain cases, removing the cross-linking agent may reduce the potential toxicity to a patient if too much of the cross-linking agent is released from shunt 26. Formed shunt 26 is preferably contacted with the quenching agent after the cross-linking treatment and, preferably, may be included with the washing/rinsing solution. Examples of quenching agents include glycine or sodium borohydride.

The formed gelatin tubes may be further treated with biologics, pharmaceuticals or other chemicals selected to regulate the body's response to the implantation of shunt 26 and the subsequent healing process. Examples of suitable agents include anti-mitolic pharmaceuticals such as Mitomycin-C or 5-Fluorouracil, anti-VEGF (such as Lucintes, Macugen, Avastin, VEGF or steroids), anti-coagulants, anti-metabolites, angiogenesis inhibitors, or steroids. The treating process can be such that only a portion of the shunt 26 is treated or an entirety of the shunt 26 is treated. For example, a portion of an exterior of shunt 26 can be treated or an entirety of an exterior of the shunt 26 can be treated. Similarly, a portion of an interior of shunt 26 can be treated or an entirety of an interior of the shunt 26 can be treated. The portion of the exterior or interior of shunt 26 to be treated may be a proximal portion, a distal portion, or a middle portion. In certain embodiments, the coated portion of shunt 26 corresponds with the portion of shunt 26 that interacts with tissue surrounded shunt 26 once it is implanted.

After the requisite drying period, the formed and cross-linked gelatin tubes are removed from the underlying supports or wires. In one embodiment, wire tubes may be cut at two ends and the formed gelatin tube slowly removed from the wire support. In another embodiment, wires with gelatin film thereon, may be pushed off using a plunger or tube to remove the formed gelatin shunt.

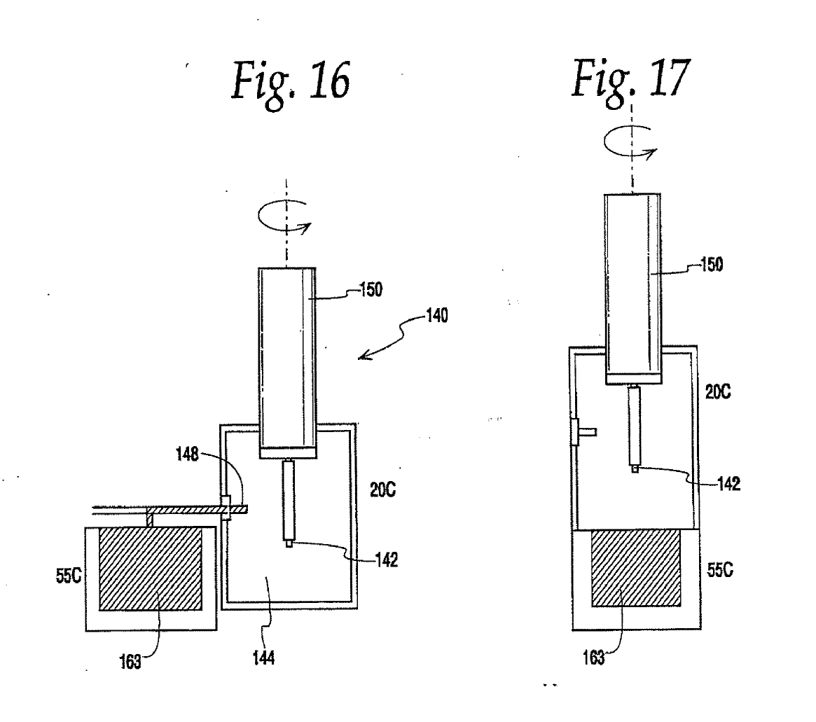

FIGS. 16 and 17 show two alternative methods and apparatus for forming gelatin shunts. In FIG. 16, apparatus 140 includes a suspended wire 142 that may be introduced into a vacuum chamber 144 at a temperature of 20° C. The gelatin solution 146 maintained at 55° C. may be applied to the wire in vacuum chamber 144 by spraying via air jet 148. Wire 142 is rotated by rotating apparatus 150 to ensure that the sprayed gelatin is applied evenly to the surface of wire 142.

In FIG. 17, a further alternative embodiment of forming gelatin tubes is shown. In accordance with the embodiment of FIG. 17, a wire 142 attached to a rotating apparatus 150 is dipped into the gelatin solution 163 at 55° C. as generally described above. Wire 142 is dipped into and removed, from the gelatin solution repeatedly and sprayed with air to ensure an even coat of the gelatin film onto the wire. In either embodiment of FIGS. 16 and 17, the gelatin tubes formed thereby may be further subjected to a cross-linking step desired above.

The gelatin tube may also be formed by preparing the mixture as described above and extruding the gelatin into a tubular shape using standard plastics processing techniques. Preparing shunt 26 by extrusion allows for providing shunts of different cross sections. For example, as shown in FIG. 34, shunts 26 having two or more passageways 260 may be provided, allowing for flow regulation. In one embodiment, passageways 260 may be selectively opened or obstructed, as shown in the shading on FIG. 34(d) to selectively control flow therethrough. One of the passageways 260 may be adapted to receive guidewire 28 or, in the alternative, shunt 26 of FIG. 34 may be used (and implanted) without a guidewire, as previously described. Shunt 26 shown in FIG. 34 may also provide greater structural integrity after implantation.

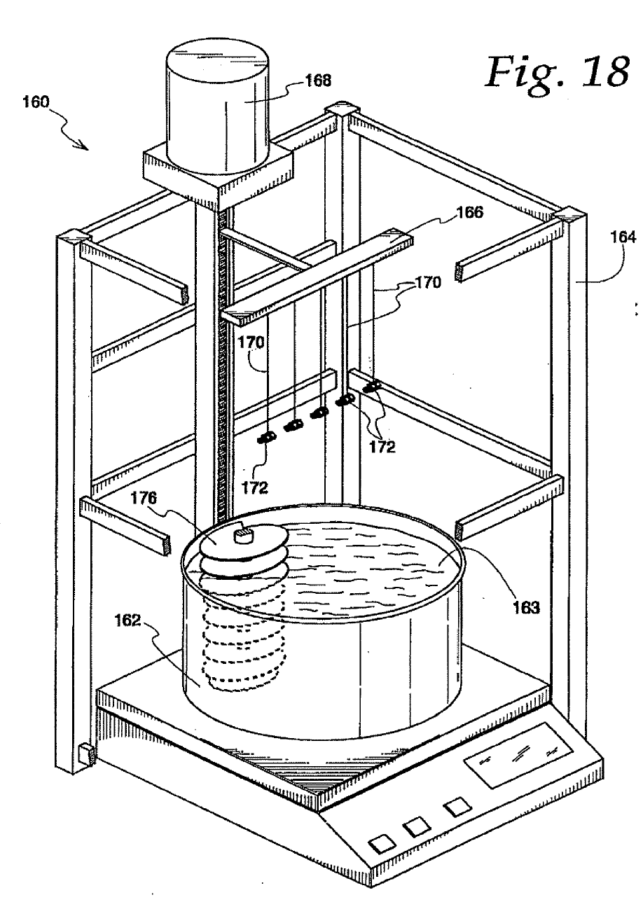

FIG. 18 shows an automated apparatus 160 for preparing a plurality of microfistula gelatin tubes. Shown in FIG. 18 is an apparatus 160 that includes a temperature controlled bath 162 of the gelatin solution 163. The apparatus includes a frame 164 that carries a vertically movable dipping arm 166. The dipping arm is coupled to a gear box 168 which is actuated by a rotary motor. The dipping arm includes a plurality of clamps (not shown) for holding several mandrel wires 170 for dipping into the gelatin solution. As further shown in FIG. 18, mandrel wires 170 may further include weights 172 suspended at their distal ends to ensure that the wire remains substantially straight (without kinking or curving) and to dampen oscillations or vibrations when being dipped in the gelatin solution 163. The operation of apparatus 160 may be controlled by a controller such as a computer with commands for dipping and withdrawal of the wires from the gelatin solution. A stirrer 176 may be provided to ensure the consistency of the gelatin solution. After the gelatin tubes have been formed, the tubes are dried and cross-linked as described above.

Shunts 26 made in accordance with the methods described above, allow for continuous and controlled drainage of aqueous humor from the anterior chamber of the eye. The preferred drainage flow rate is approximately 2 microliters per minute, although by varying the inner diameter and length of shunt 26, the flow rate may be adjusted as needed. One or more shunts 26 may be implanted into the eye of the patient to further control the drainage.

In addition to providing a safe and efficient way to relieve intraocular pressure in the eye, it has been observed that implanted shunts disclosed herein can also contribute to regulating the flow rate (due to resistance of the lymphatic outflow tract) and stimulate growth of functional drainage structures between the eye and the lymphatic and/or venous systems. These drainage structures evacuate fluid from the subconjunctiva which also result in a low diffuse bleb, a small bleb reservoir or no bleb whatsoever.

The formation of drainage pathways formed by and to the lymphatic system and/or veins may have applications beyond the treatment of glaucoma. Thus, the methods of shunt implantation may be useful in the treatment of other tissues and organs where drainage may be desired or required.

In addition, it has been observed that as the microfistula shunt absorbs, a “natural” microfistula shunt or pathway lined with cells is formed. This “natural” shunt is stable. The implanted shunt stays in place (thereby keeping the opposing sides of the formed shunt separated) long enough to allow for a confluent covering of cells to form. Once these cells form, they are stable, thus eliminating the need for a foreign body to be placed in the formed space.

### Tissue Compatible Shunts

In certain aspects, the invention generally provides shunts composed of a material that has an elasticity modulus that is compatible with an elasticity modulus of tissue surrounding the shunt. In this manner, shunts of the invention are flexibility matched with the surrounding tissue, and thus will remain in place after implantation without the need for any type of anchor that interacts with the surrounding tissue. Consequently, shunts of the invention will maintain fluid flow away for an anterior chamber of the eye after implantation without causing irritation or inflammation to the tissue surrounding the eye.

Elastic modulus, or modulus of elasticity, is a mathematical description of an object or substance's tendency to be deformed elastically when a force is applied to it. The elastic modulus of an object is defined as the slope of its stress-strain curve in the elastic deformation region:

$$λ {=}^{def} \frac{stress}{strain}$$

where lambda (λ) is the elastic modulus; stress is the force causing the deformation divided by the area to which the force is applied; and strain is the ratio of the change caused by the stress to the original state of the object. The elasticity modulus may also be known as Young's modulus (E), which describes tensile elasticity, or the tendency of an object to deform along an axis when opposing forces are applied along that axis. Young's modulus is defined as the ratio of tensile stress to tensile strain. For further description regarding elasticity modulus and Young's modulus, see for example Gere (Mechanics of Materials, 6^{th} Edition, 2004, Thomson), the content of which is incorporated by reference herein in its entirety.

The elasticity modulus of any tissue can be determined by one of skill in the art. See for example Samani et al. (Phys. Med. Biol. 48:2183, 2003); Erkamp et al. (Measuring The Elastic Modulus Of Small Tissue Samples, Biomedical Engineering Department and Electrical Engineering and Computer Science Department University of Michigan Ann Arbor, Mich. 48109-2125; and Institute of Mathematical Problems in Biology Russian Academy of Sciences, Pushchino, Moscow Region 142292 Russia); Chen et al. (IEEE Trans. Ultrason. Ferroelec. Freq. Control 43:191-194, 1996); Hall, (In 1996 Ultrasonics Symposium Proc., pp. 1193-1196, IEEE Cat. No. 96CH35993, IEEE, New York, 1996); and Parker (Ultrasound Med. Biol. 16:241-246, 1990), each of which provides methods of determining the elasticity modulus of body tissues. The content of each of these is incorporated by reference herein in its entirety.

The elasticity modulus of tissues of different organs is known in the art. For example, Pierscionek et al. (Br J Ophthalmol, 91:801-803, 2007) and Friberg (Experimental Eye Research, 473:429-436, 1988) show the elasticity modulus of the cornea and the sclera of the eye. The content of each of these references is incorporated by reference herein in its entirety. Chen, Hall, and Parker show the elasticity modulus of different muscles and the liver. Erkamp shows the elasticity modulus of the kidney.

Shunts of the invention are composed of a material that is compatible with an elasticity modulus of tissue surrounding the shunt. In certain embodiments, the material has an elasticity modulus that is substantially identical to the elasticity modulus of the tissue surrounding the shunt. In other embodiments, the material has an elasticity modulus that is greater than the elasticity modulus of the tissue surrounding the shunt. Exemplary materials includes biocompatible polymers, such as polycarbonate, polyethylene, polyethylene terephthalate, polyimide, polystyrene, polypropylene, poly(styrene-b-isobutylene-b-styrene), or silicone rubber.

In particular embodiments, shunts of the invention are composed of a material that has an elasticity modulus that is compatible with the elasticity modulus of tissue in the eye, particularly scleral tissue. In certain embodiments, compatible materials are those materials that are softer than scleral tissue or marginally harder than scleral tissue, yet soft enough to prohibit shunt migration. The elasticity modulus for anterior scleral tissue is approximately 2.9±1.4×10⁶ N/m², and 1.8±1.1×10⁶ N/m² for posterior scleral tissue. See Friberg (Experimental Eye Research, 473:429-436, 1988). An exemplary material is cross linked gelatin derived from Bovine or Porcine Collagen.

The invention encompasses shunts of different shapes and different dimensions, and the shunts of the invention may be any shape or any dimension that may be accommodated by the eye. In certain embodiments, the intraocular shunt is of a cylindrical shape and has an outside cylindrical wall and a hollow interior. The shunt may have an inside diameter from approximately 10 μm to approximately 250 μm, an outside diameter from approximately 80 μm to approximately 300 μm, and a length from approximately 0.5 mm to approximately 20 mm.

Shunts of the invention may be impregnated or treated with biologics, pharmaceuticals or other chemicals selected to regulate the body's response to the implantation of the shunt and the subsequent healing process. Examples of suitable agents include anti-mitolic pharmaceuticals such as Mitomycin-C or 5-Fluorouracil, anti-VEGF (such as Lucintes, Macugen, Avastin, VEGF or steroids), anti-coagulants, anti-metabolites, angiogenesis inhibitors, or steroids. By including the biologics, pharmaceuticals or other chemicals in the liquid gelatin, the formed shunt will be impregnated with the biologics, pharmaceuticals or other chemicals. The treating process can be such that only a portion of the shunt is treated or an entirety of the shunt is treated. For example, a portion of an exterior of the shunt can be treated or an entirety of an exterior of the shunt can be treated. Similarly, a portion of an interior of the shunt can be treated or an entirety of an interior of the shunt can be treated. The portion of the exterior or interior of the shunt to be treated may be a proximal portion, a distal portion, or a middle portion. In certain embodiments, the coated portion of the shunt corresponds with the portion of the shunt that interacts with tissue surrounded the shunt once it is implanted.

### Shunts Reactive to Pressure

In other aspects, the invention generally provides shunts in which a portion of the shunt is composed of a flexible material that is reactive to pressure, i.e., the diameter of the flexible portion of the shunt fluctuates depending upon the pressures exerted on that portion of the shunt. An exemplary shunt of these embodiments is a shunt in which flexible portion is the middle portion. However, the flexible portion may be located in any portion of the shunt, such as the proximal or distal portion of the shunt. In certain embodiments, the entire shunt is composed of the flexible material, and thus the entire shunt is flexible and reactive to pressure.

The flexible portion of the shunt acts as a valve that regulates fluid flow through the shunt. The human eye produces aqueous humor at a rate of about 2 μl/min for approximately 3 ml/day. The entire aqueous volume is about 0.25 ml. When the pressure in the anterior chamber falls after surgery to about 7-8 mmHg, it is assumed the majority of the aqueous humor is exiting the eye through the implant since venous backpres sure prevents any significant outflow through normal drainage structures (e.g., the trabecular meshwork).

After implantation, intraocular shunts have pressure exerted upon them by tissues surrounding the shunt (e.g., scleral tissue such as the sclera shunt and the sclera exit) and pressure exerted upon them by aqueous humor flowing through the shunt. The flow through the shunt, and thus the pressure exerted by the fluid on the shunt, is calculated by the equation:

$$Φ = \frac{d V}{d t} = v π {R}^{2} = \frac{π {R}^{4}}{8 η} ( \frac{- Δ P}{Δ x} ) = \frac{π {R}^{4}}{8 η} \frac{\vert Δ P \vert}{L}$$

where Φ is the volumetric flow rate; V is a volume of the liquid poured (cubic meters); t is the time (seconds); v is mean fluid velocity along the length of the tube (meters/second); x is a distance in direction of flow (meters); R is the internal radius of the tube (meters); ΔP is the pressure difference between the two ends (pascals); η is the dynamic fluid viscosity (pascal-second (Pa·s)); and L is the total length of the tube in the x direction (meters).

Shunts of these embodiments, may be implanted into an eye for regulation of fluid flow from the anterior chamber of the eye to an area of lower pressure (e.g., intra-Tenon's space, the subconjunctival space, the episcleral vein, the suprachoroidal space, or Schlemm's canal). In certain embodiments, the area of lower pressure is the subarachnoid space. The shunt is implanted such that a proximal end of the shunt resides in the anterior chamber of the eye, and a distal end of the shunt resides outside of the anterior chamber to conduct aqueous humor from the anterior chamber to an area of lower pressure. A flexible portion of the shunt spans at least a portion of the sclera of the eye, e.g., the flexible portion spans an entire length of the sclera.

When the pressure exerted on the flexible portion of the shunt by sclera is greater than the pressure exerted on the flexible portion of the shunt by the fluid flowing through the shunt, the flexible portion decreases in diameter, restricting flow through the shunt. The restricted flow results in aqueous humor leaving the anterior chamber at a reduced rate.

When the pressure exerted on the flexible portion of the shunt by the fluid flowing through the shunt is greater than the pressure exerted on the flexible portion of the shunt by the sclera, the flexible portion increases in diameter, increasing flow through the shunt. The increased flow results in aqueous humor leaving the anterior chamber at an increased rate.

The invention encompasses shunts of different shapes and different dimensions, and the shunts of the invention may be any shape or any dimension that may be accommodated by the eye. In certain embodiments, the intraocular shunt is of a cylindrical shape and has an outside cylindrical wall and a hollow interior. The shunt may have an inside diameter from approximately 10 μm to approximately 250 μm, an outside diameter from approximately 80 μm to approximately 300 μm, and a length from approximately 0.5 mm to approximately 20 mm.

In a particular embodiments, the shunt has a length of about 6 mm and an inner diameter of about 64 μm. With these dimensions, the pressure difference between the proximal end of the shunt that resides in the anterior chamber and the distal end of the shunt that resides outside the anterior chamber is about 4.3 mmHg. Such dimensions thus allow the implant to act as a controlled valve and protect the integrity of the anterior chamber.

It will be appreciated that different dimensioned implants may be used. For example, shunts that range in length from about 0.5 mm to about 20 mm and have a range in inner diameter from about 10 μm to about 100 μm allow for pressure control from approximately 0.5 mmHg to approximately 20 mmHg.

The material of the flexible portion and the thickness of the wall of the flexible portion will determine how reactive the flexible portion is to the pressures exerted upon it by the surrounding tissue and the fluid flowing through the shunt. Generally, with a certain material, the thicker the flexible portion, the less responsive the portion will be to pressure. In certain embodiments, the flexible portion is a gelatin or other similar material, and the thickness of the gelatin material forming the wall of the flexible portion ranges from about 10 μm thick to about 100 μm thick.

In a certain embodiment, the gelatin used for making the flexible portion is known as gelatin Type B from bovine skin. An exemplary gelatin is PB Leiner gelatin from bovine skin, Type B, 225 Bloom, USP. Another material that may be used in the making of the flexible portion is a gelatin Type A from porcine skin, also available from Sigma Chemical. Such gelatin is available from Sigma Chemical Company of St. Louis, Mo. under Code G-9382. Still other suitable gelatins include bovine bone gelatin, porcine bone gelatin and human-derived gelatins. In addition to gelatins, the flexible portion may be made of hydroxypropyl methycellulose (HPMC), collagen, polylactic acid, polylglycolic acid, hyaluronic acid and glycosaminoglycans.

In certain embodiments, the gelatin is cross-linked. Cross-linking increases the inter- and intramolecular binding of the gelatin substrate. Any method for cross-linking the gelatin may be used. In a particular embodiment, the formed gelatin is treated with a solution of a cross-linking agent such as, but not limited to, glutaraldehyde. Other suitable compounds for cross-linking include 1-ethyl-3-[3-(dimethyamino)propyl]carbodiimide (EDC). Cross-linking by radiation, such as gamma or electron beam (e-beam) may be alternatively employed.

In one embodiment, the gelatin is contacted with a solution of approximately 25% glutaraldehyde for a selected period of time. One suitable form of glutaraldehyde is a grade 1G5882 glutaraldehyde available from Sigma Aldridge Company of Germany, although other glutaraldehyde solutions may also be used. The pH of the glutaraldehyde solution should be in the range of 7 to 7.8 and, more particularly, 7.35-7.44 and typically approximately 7.4+/−0.01. If necessary, the pH may be adjusted by adding a suitable amount of a base such as sodium hydroxide as needed.

Methods for forming the flexible portion of the shunt are shown for example in Yu et al. (U.S. patent application number 2008/0108933), the content of which is incorporated by reference herein in its entirety. In an exemplary protocol, the flexible portion may be made by dipping a core or substrate such as a wire of a suitable diameter in a solution of gelatin. The gelatin solution is typically prepared by dissolving a gelatin powder in de-ionized water or sterile water for injection and placing the dissolved gelatin in a water bath at a temperature of approximately 55° C. with thorough mixing to ensure complete dissolution of the gelatin. In one embodiment, the ratio of solid gelatin to water is approximately 10% to 50% gelatin by weight to 50% to 90% by weight of water. In an embodiment, the gelatin solution includes approximately 40% by weight, gelatin dissolved in water. The resulting gelatin solution should be devoid of air bubbles and has a viscosity that is between approximately 200-500 cp and more particularly between approximately 260 and 410 cp (centipoise).

Once the gelatin solution has been prepared, in accordance with the method described above, supporting structures such as wires having a selected diameter are dipped into the solution to form the flexible portion. Stainless steel wires coated with a biocompatible, lubricious material such as polytetrafluoroethylene (Teflon) are preferred.

Typically, the wires are gently lowered into a container of the gelatin solution and then slowly withdrawn. The rate of movement is selected to control the thickness of the coat. In addition, it is preferred that a the tube be removed at a constant rate in order to provide the desired coating. To ensure that the gelatin is spread evenly over the surface of the wire, in one embodiment, the wires may be rotated in a stream of cool air which helps to set the gelatin solution and affix film onto the wire. Dipping and withdrawing the wire supports may be repeated several times to further ensure even coating of the gelatin. Once the wires have been sufficiently coated with gelatin, the resulting gelatin films on the wire may be dried at room temperature for at least 1 hour, and more preferably, approximately 10 to 24 hours. Apparatus for forming gelatin tubes are described in Yu et al. (U.S. patent application number 2008/0108933).

Once dried, the formed flexible portions may be treated with a cross-linking agent. In one embodiment, the formed flexible portion may be cross-linked by dipping the wire (with film thereon) into the 25% glutaraldehyde solution, at pH of approximately 7.0-7.8 and more preferably approximately 7.35-7.44 at room temperature for at least 4 hours and preferably between approximately 10 to 36 hours, depending on the degree of cross-linking desired. In one embodiment, the formed flexible portion is contacted with a cross-linking agent such as gluteraldehyde for at least approximately 16 hours. Cross-linking can also be accelerated when it is performed a high temperatures. It is believed that the degree of cross-linking is proportional to the bioabsorption time of the shunt once implanted. In general, the more cross-linking, the longer the survival of the shunt in the body.

The residual glutaraldehyde or other cross-linking agent is removed from the formed flexible portion by soaking the tubes in a volume of sterile water for injection. The water may optionally be replaced at regular intervals, circulated or re-circulated to accelerate diffusion of the unbound glutaraldehyde from the tube. The tubes are washed for a period of a few hours to a period of a few months with the ideal time being 3-14 days. The now cross-linked gelatin tubes may then be dried (cured) at ambient temperature for a selected period of time. It has been observed that a drying period of approximately 48-96 hours and more typically 3 days (i.e., 72 hours) may be preferred for the formation of the cross-linked gelatin tubes.

Where a cross-linking agent is used, it may be desirable to include a quenching agent in the method of making the flexible portion. Quenching agents remove unbound molecules of the cross-linking agent from the formed flexible portion. In certain cases, removing the cross-linking agent may reduce the potential toxicity to a patient if too much of the cross-linking agent is released from the flexible portion. In certain embodiments, the formed flexible portion is contacted with the quenching agent after the cross-linking treatment and, may be included with the washing/rinsing solution. Examples of quenching agents include glycine or sodium borohydride.

The formed flexible portion may be further coated or impregnated with biologics and/or pharmaceuticals. Any pharmaceutical and/or biological agent or combination thereof may be used with shunts of the invention. The pharmaceutical and/or biological agent may be released over a short period of time (e.g., seconds) or may be released over longer periods of time (e.g., days, weeks, months, or even years). In certain embodiments, the pharmaceutical and/or biological agent is selected to regulate the body's response to the implantation of the implant and assist in the subsequent healing process. Exemplary agents include anti-mitotic pharmaceuticals such as Mitomycin-C or 5-Fluorouracil, anti-VEGF (such as Lucintes, Macugen, Avastin, VEGF or steroids).

After the requisite drying period, the formed and cross-linked flexible portion is removed from the underlying supports or wires. In one embodiment, wire tubes may be cut at two ends and the formed gelatin flexible portion slowly removed from the wire support. In another embodiment, wires with gelatin film thereon, may be pushed off using a plunger or tube to remove the formed gelatin flexible portion.

Shunts of the invention may be impregnated or treated with biologics, pharmaceuticals or other chemicals selected to regulate the body's response to the implantation of the shunt and the subsequent healing process. Examples of suitable agents include anti-mitolic pharmaceuticals such as Mitomycin-C or 5-Fluorouracil, anti-VEGF (such as Lucintes, Macugen, Avastin, VEGF or steroids), anti-coagulants, anti-metabolites, angiogenesis inhibitors, or steroids. By including the biologics, pharmaceuticals or other chemicals in the liquid gelatin, the formed shunt will be impregnated with the biologics, pharmaceuticals or other chemicals. The treating process can be such that only a portion of the shunt is treated or an entirety of the shunt is treated. For example, a portion of an exterior of the shunt can be treated or an entirety of an exterior of the shunt can be treated. Similarly, a portion of an interior of the shunt can be treated or an entirety of an interior of the shunt can be treated. The portion of the exterior or interior of the shunt to be treated may be a proximal portion, a distal portion, or a middle portion. In certain embodiments, the coated portion of the shunt corresponds with the portion of the shunt that interacts with tissue surrounded the shunt once it is implanted.

### Multi-Port Shunts

Other aspects of the invention generally provide multi-port shunts. Such shunts reduce probability of the shunt clogging after implantation because fluid can enter or exit the shunt even if one or more ports of the shunt become clogged with particulate. In certain embodiments, the shunt includes a hollow body defining a flow path and more than two ports, in which the body is configured such that a proximal portion receives fluid from the anterior chamber of an eye and a distal portion directs the fluid to a location of lower pressure with respect to the anterior chamber. Exemplary areas of lower pressure include intra-Tenon's space, the subconjunctival space, the episcleral vein, the suprachoroidal space, or Schlemm's canal. Another exemplary area of lower pressure to which fluid may be drained in the subarachnoid space.

The shunt may have many different configurations. An exemplary multi-port shunt is one in which the proximal portion of the shunt (i.e., the portion disposed within the anterior chamber of the eye) includes more than one port and the distal portion of the shunt (i.e., the portion that is located near a drainage structure such as) includes a single port. Another exemplary multi-port shunt is one in which the proximal portion includes a single port and the distal portion includes more than one port. Another exemplary multi-port shunt is one in which the proximal portions include more than one port and the distal portions include more than one port. Multi-port shunts of the invention may include any number of ports at either the proximal or distal end. For example, shunts of the invention may include five ports at the proximal portion, distal portion, or both, those shunts are only exemplary embodiments. The ports may be located along any portion of the shunt, and shunts of the invention include all shunts having more than two ports. For example, shunts of the invention may include at least three ports, at least four ports, at least five ports, at least 10 ports, at least 15 ports, or at least 20 ports.

The ports may be positioned in various different orientations and along various different portions of the shunt. In certain embodiments, at least one of the ports is oriented at an angle to the length of the body. In certain embodiments, at least one of the ports is oriented 90° to the length of the body. The ports may have the same or different inner diameters. In certain embodiments, at least one of the ports has an inner diameter that is different from the inner diameters of the other ports. The inner diameters of the ports may range from about 20 μm to about 40 μm, particularly about 30 μm.

The invention encompasses shunts of different shapes and different dimensions, and the shunts of the invention may be any shape or any dimension that may be accommodated by the eye. In certain embodiments, the intraocular shunt is of a cylindrical shape and has an outside cylindrical wall and a hollow interior. The shunt may have an inside diameter from approximately 10 μm to approximately 250 μm, an outside diameter from approximately 80 μm to approximately 300 μm, and a length from approximately 0.5 mm to approximately 20 mm. Shunts of the invention may be made from any biocompatible material. An exemplary material is gelatin. Methods of making shunts composed of gelatin are described above.

Shunts of the invention may be impregnated or treated with biologics, pharmaceuticals or other chemicals selected to regulate the body's response to the implantation of the shunt and the subsequent healing process. Examples of suitable agents include anti-mitolic pharmaceuticals such as Mitomycin-C or 5-Fluorouracil, anti-VEGF (such as Lucintes, Macugen, Avastin, VEGF or steroids), anti-coagulants, anti-metabolites, angiogenesis inhibitors, or steroids. By including the biologics, pharmaceuticals or other chemicals in the liquid gelatin, the formed shunt will be impregnated with the biologics, pharmaceuticals or other chemicals. The treating process can be such that only a portion of the shunt is treated or an entirety of the shunt is treated. For example, a portion of an exterior of the shunt can be treated or an entirety of an exterior of the shunt can be treated. Similarly, a portion of an interior of the shunt can be treated or an entirety of an interior of the shunt can be treated. The portion of the exterior or interior of the shunt to be treated may be a proximal portion, a distal portion, or a middle portion. In certain embodiments, the coated portion of the shunt corresponds with the portion of the shunt that interacts with tissue surrounded the shunt once it is implanted.

Shunts with Overflow Ports

Other aspects of the invention generally provide shunts with overflow ports. Those shunts are configured such that the overflow port remains partially or completely closed until there is a pressure build-up within the shunt sufficient to force open the overflow port. Such pressure build-up typically results from particulate partially or fully clogging an entry or an exit port of the shunt. Such shunts reduce probability of the shunt clogging after implantation because fluid can enter or exit the shunt by the overflow port even in one port of the shunt becomes clogged with particulate.

In certain embodiments, the shunt includes a hollow body defining an inlet configured to receive fluid from an anterior chamber of an eye and an outlet configured to direct the fluid to a location of lower pressure with respect to the anterior chamber, the body further including at least one slit. The slit may be located at any place along the body of the shunt. An exemplary shunt is a shunt having an inlet, an outlet, and a slit located in proximity to the inlet. Another exemplary embodiment includes a shunt having an inlet, an outlet, and a slit located in proximity to the outlet. Another exemplary embodiment includes a shunt having an inlet, an outlet, a slit located in proximity to the inlet, and a slit located in proximity to the outlet.

The overflow port(s) may be located along any portion of the shunt, and shunts of the invention include shunts having more than one overflow port. In certain embodiments, shunts of the invention include more than one overflow port at the proximal portion, the distal portion, or both. For example, a shunt may include an inlet, an outlet, and two slits located in proximity to the inlet. Shunts of the invention may include at least two overflow ports, at least three overflow ports, at least four overflow ports, at least five overflow ports, at least 10 overflow ports, at least 15 overflow ports, or at least 20 overflow ports. In certain embodiments, shunts of the invention include two slits that overlap and are oriented at 90° to each other, thereby forming a cross. In certain embodiments, the slit may be at the proximal or the distal end of the shunt, producing a split in the proximal or the distal end of the implant.

In certain embodiments, the slit has a width that is substantially the same or less than an inner diameter of the inlet. In other embodiments, the slit has a width that is substantially the same or less than an inner diameter of the outlet. In certain embodiments, the slit has a length that ranges from about 0.05 mm to about 2 mm, and a width that ranges from about 10 μm to about 200 μm. Generally, the slit does not direct the fluid unless the outlet is obstructed. However, the shunt may be configured such that the slit does direct at least some of the fluid even if the inlet or outlet is not obstructed.

The invention encompasses shunts of different shapes and different dimensions, and the shunts of the invention may be any shape or any dimension that may be accommodated by the eye. In certain embodiments, the intraocular shunt is of a cylindrical shape and has an outside cylindrical wall and a hollow interior. The shunt may have an inside diameter from approximately 10 μm to approximately 250 μm, an outside diameter from approximately 80 μm to approximately 300 μm, and a length from approximately 0.5 mm to approximately 20 mm. Shunts of the invention may be made from any biocompatible material. An exemplary material is gelatin. Methods of making shunts composed of gelatin are described above.

Shunts of the invention may be impregnated or treated with biologics, pharmaceuticals or other chemicals selected to regulate the body's response to the implantation of the shunt and the subsequent healing process. Examples of suitable agents include anti-mitolic pharmaceuticals such as Mitomycin-C or 5-Fluorouracil, anti-VEGF (such as Lucintes, Macugen, Avastin, VEGF or steroids), anti-coagulants, anti-metabolites, angiogenesis inhibitors, or steroids. By including the biologics, pharmaceuticals or other chemicals in the liquid gelatin, the formed shunt will be impregnated with the biologics, pharmaceuticals or other chemicals. The treating process can be such that only a portion of the shunt is treated or an entirety of the shunt is treated. For example, a portion of an exterior of the shunt can be treated or an entirety of an exterior of the shunt can be treated. Similarly, a portion of an interior of the shunt can be treated or an entirety of an interior of the shunt can be treated. The portion of the exterior or interior of the shunt to be treated may be a proximal portion, a distal portion, or a middle portion. In certain embodiments, the coated portion of the shunt corresponds with the portion of the shunt that interacts with tissue surrounded the shunt once it is implanted.

### Shunts Having a Variable Inner Diameter

In other aspects, the invention generally provides a shunt having a variable inner diameter. In particular embodiments, the diameter increases from inlet to outlet of the shunt. By having a variable inner diameter that increases from inlet to outlet, a pressure gradient is produced and particulate that may otherwise clog the inlet of the shunt is forced through the inlet due to the pressure gradient. Further, the particulate will flow out of the shunt because the diameter only increases after the inlet.

An exemplary shunt includes an inlet configured to receive fluid from an anterior chamber of an eye and an outlet configured to direct the fluid to a location of lower pressure with respect to the anterior chamber, in which the body further includes a variable inner diameter that increases along the length of the body from the inlet to the outlet. In certain embodiments, the inner diameter continuously increases along the length of the body. In other embodiments, the inner diameter remains constant along portions of the length of the body.

In exemplary embodiments, the inner diameter may range in size from about 10 μm to about 200 μm, and the inner diameter at the outlet may range in size from about 15 μm to about 300 μm. The invention encompasses shunts of different shapes and different dimensions, and the shunts of the invention may be any shape or any dimension that may be accommodated by the eye. In certain embodiments, the intraocular shunt is of a cylindrical shape and has an outside cylindrical wall and a hollow interior. The shunt may have an inside diameter from approximately 10 μm to approximately 250 μm, an outside diameter from approximately 80 μm to approximately 300 μm, and a length from approximately 0.5 mm to approximately 20 mm. Shunts of the invention may be made from any biocompatible material. An exemplary material is gelatin. Methods of making shunts composed of gelatin are described above.

Shunts of the invention may be impregnated or treated with biologics, pharmaceuticals or other chemicals selected to regulate the body's response to the implantation of the shunt and the subsequent healing process. Examples of suitable agents include anti-mitolic pharmaceuticals such as Mitomycin-C or 5-Fluorouracil, anti-VEGF (such as Lucintes, Macugen, Avastin, VEGF or steroids), anti-coagulants, anti-metabolites, angiogenesis inhibitors, or steroids. By including the biologics, pharmaceuticals or other chemicals in the liquid gelatin, the formed shunt will be impregnated with the biologics, pharmaceuticals or other chemicals. The treating process can be such that only a portion of the shunt is treated or an entirety of the shunt is treated. For example, a portion of an exterior of the shunt can be treated or an entirety of an exterior of the shunt can be treated. Similarly, a portion of an interior of the shunt can be treated or an entirety of an interior of the shunt can be treated. The portion of the exterior or interior of the shunt to be treated may be a proximal portion, a distal portion, or a middle portion. In certain embodiments, the coated portion of the shunt corresponds with the portion of the shunt that interacts with tissue surrounded the shunt once it is implanted.

### Shunts Having Pronged Ends

In other aspects, the invention generally provides shunts for facilitating conduction of fluid flow away from an organ, the shunt including a body, in which at least one end of the shunt is shaped to have a plurality of prongs. Such shunts reduce probability of the shunt clogging after implantation because fluid can enter or exit the shunt by any space between the prongs even if one portion of the shunt becomes clogged with particulate.

In certain embodiments, at least one end of these shunts includes a plurality of prongs. In other embodiments, both a proximal end and a distal end of the shunt are shaped to have the plurality of prongs. However, numerous different configurations are envisioned. For example, in certain embodiments, only the proximal end of the shunt is shaped to have the plurality of prongs. In other embodiments, only the distal end of the shunt is shaped to have the plurality of prongs.

The prongs can have any shape (i.e., width, length, height). For example, the prongs may be straight prongs. In this embodiment, the spacing between the prongs is the same. In another embodiment, the prongs are tapered. In this embodiment, the spacing between the prongs increases toward a proximal and/or distal end of the shunt.

In a particular embodiment, the shunt includes four prongs. However, shunts of the invention may accommodate any number of prongs, such as two prongs, three prongs, four prongs, five prongs, six prongs, seven prongs, eight prongs, nine prongs, ten prongs, etc. The number of prongs chosen will depend on the desired flow characteristics of the shunt.

The invention encompasses shunts of different shapes and different dimensions, and the shunts of the invention may be any shape or any dimension that may be accommodated by the eye. In certain embodiments, the intraocular shunt is of a cylindrical shape and has an outside cylindrical wall and a hollow interior. The shunt may have an inside diameter from approximately 10 μm to approximately 250 μm, an outside diameter from approximately 80 μm to approximately 300 μm, and a length from approximately 0.5 mm to approximately 20 mm. Shunts of the invention may be made from any biocompatible material. An exemplary material is gelatin. Methods of making shunts composed of gelatin are described above.

### Shunts Having a Longitudinal Slit

In other aspects, the invention generally provides a shunt for draining fluid from an anterior chamber of an eye that includes a hollow body defining an inlet configured to receive fluid from an anterior chamber of the eye and an outlet configured to direct the fluid to a location of lower pressure with respect to the anterior chamber; the shunt being configured such that at least one end of the shunt includes a longitudinal slit. Such shunts reduce probability of the shunt clogging after implantation because the end(s) of the shunt can more easily pass particulate which would generally clog a shunt lacking the slits.

In certain embodiments, at least one end of these shunts includes a longitudinal slit that produces a top portion and a bottom portion in a proximal and/or distal end of the shunt. In other embodiments, both a proximal end and a distal end include a longitudinal slit that produces a top portion and a bottom portion in both ends of the shunt. However, numerous different configurations are envisioned. For example, in certain embodiments, only the proximal end of the shunt includes a longitudinal slit. In other embodiments, only the distal end of the shunt includes a longitudinal slit.

The longitudinal slit can have any shape (i.e., width, length, height). For example, the longitudinal slit can be straight such that the space between the top portion and the bottom portion remains the same along the length of the slit. In another embodiment, the longitudinal slit is tapered. In this embodiment, the space between the top portion and the bottom portion increases toward a proximal and/or distal end of the shunt.

The invention encompasses shunts of different shapes and different dimensions, and the shunts of the invention may be any shape or any dimension that may be accommodated by the eye. In certain embodiments, the intraocular shunt is of a cylindrical shape and has an outside cylindrical wall and a hollow interior. The shunt may have an inside diameter from approximately 10 μm to approximately 250 μm, an outside diameter from approximately 80 μm to approximately 300 μm, and a length from approximately 0.5 mm to approximately 20 mm. Shunts of the invention may be made from any biocompatible material. An exemplary material is gelatin. Methods of making shunts composed of gelatin are described above.

### Combinations of Embodiments

As will be appreciated by one skilled in the art, individual features of the invention may be used separately or in any combination. Particularly, it is contemplated that one or more features of the individually described above embodiments may be combined into a single shunt.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

What is claimed is:

1. A shunt for draining fluid from an anterior chamber of an eye, the shunt comprising: a hollow body defining a flow path and having an inlet configured to receive fluid from an anterior chamber of an eye and an outlet configured to direct the fluid to a location of lower pressure with respect to the anterior chamber, wherein at least a portion of the body comprises a drug.

2. The shunt according to claim 1, wherein the shunt is made of gelatin.

3. The shunt according to claim 2, wherein the drug is impregnated in the body of the shunt.

4. The shunt according to claim 1, wherein the drug coats at least a portion of an exterior of the shunt.

5. The shunt according to claim 4, wherein the coated portion corresponds with the portion of the shunt that interacts with tissue surrounded the shunt once it is implanted.

6. The shunt according to claim 4, wherein a proximal portion of the shunt is coated.

7. The shunt according to claim 4, wherein a distal portion of the shunt is coated.

8. The shunt according to claim 4, wherein a middle portion of the shunt is coated.

9. The shunt according to claim 4, wherein the drug coats an entirety of an exterior of the shunt.

10. The shunt according to claim 1, wherein the drug coats at least a portion of an interior of the shunt.

11. The shunt according to claim 1, wherein the drug coats an entirety of an interior of the shunt.

12. The shunt according to claim 4, wherein the drug is selected from the group consisting of: an anticoagulant, an antimetabolite, an angiogenesis inhibitor, and a steroid.

13. The shunt according to claim 1, wherein the location is selected from the group consisting of: intra-Tenon's space, the subconjunctival space, the episcleral vein, the suprachoroidal space, and Schlemm's canal.

14. The shunt according to claim 1, wherein the shunt comprising a material that has an elasticity modulus that is compatible with an elasticity modulus of tissue surrounding the shunt.

15. The shunt according to claim 14, wherein the material has an elasticity modulus that is substantially identical to the elasticity modulus of the tissue surrounding the shunt.

16. The shunt according to claim 14, wherein the material has an elasticity modulus that is greater than the elasticity modulus of the tissue surrounding the shunt.

17. The shunt according to claim 1, wherein at least a portion of the body is comprised of a flexible material that allows for fluctuation of an inner diameter of the portion of the shaft based upon pressure exerted from surrounding tissue and/or fluid in the organ.

18. The shunt according to claim 17, wherein the portion of the body that is comprised of the flexible material is a distal portion of the body.

19. The shunt according to claim 17, wherein the portion of the body that is comprised of the flexible material is a middle portion of the body.

20. The shunt according to claim 17, wherein the entire shaft comprises the flexible material.

21. The shunt according to claim 1, wherein the body comprises more than two ports.

22. The shunt according to claim 21, wherein the proximal portion comprises more than one port and the distal portion comprises a single port.

23. The shunt according to claim 21, wherein the proximal portion comprises a single port and the distal portion comprises more than one port.

24. The shunt according to claim 21, wherein the proximal and the distal portions comprise more than one port.

25. The shunt according to claim 21, wherein at least one of the ports is oriented 90° to the length of the body.

26. The shunt according to claim 21, wherein at least one of the ports is oriented at an angle to the length of the body.

27. The shunt according to claim 1, wherein the body comprises at least one slit.

28. The shunt according to claim 27, wherein the slit is located in proximity to the inlet.

29. The shunt according to claim 28, wherein the slit has a width that is substantially the same or less than an inner diameter of the inlet.

30. The shunt according to claim 27, wherein the slit is located in proximity to the outlet.

31. The shunt according to claim 29, wherein the slit has a width that is substantially the same or less than an inner diameter of the outlet.

32. The shunt according to claim 30, wherein the slit does not direct the fluid unless the outlet is obstructed.

33. The shunt according to claim 27, wherein both the inlet and the outlet comprise a slit.

34. The shunt according to claim 1, wherein the body comprises a variable inner diameter that increases along the length of the body length from the inlet to the outlet.

35. The shunt according to claim 34, wherein the inner diameter continuously increases along the length of the body.

36. The shunt according to claim 34, wherein the inner diameter remains constant along portions of the length of the body.

37. The shunt according to claim 1, wherein the shunt is bioabsorbable.

38. The shunt according to claim 1, wherein at least one end of the shunt is shaped to have a plurality of prongs.

39. The shunt according to claim 38, wherein a proximal end of the shunt is shaped to have the plurality of prongs.

40. The shunt according to claim 38, wherein a distal end of the shunt is shaped to have the plurality of prongs.

41. The shunt according to claim 38, wherein both a proximal end and a distal end of the shunt are shaped to have the plurality of prongs.

42. The shunt according to claim 1, wherein at least one end of the shunt comprises a longitudinal slit.

43. The shunt according to claim 42, wherein the slit is at a proximal end of the shunt.

44. The shunt according to claim 42, wherein the slit is at a distal end of the shunt.

45. The shunt according to claim 42, wherein both a proximal end and a distal end of the shunt comprise the slits.

46. The shunt according to claim 42, wherein the slit has a width that is substantially the same or less than an inner diameter of the inlet or outlet.
